(19) United States
(12) Patent Application Publication
(10) Pub. No.: US 2026/0346426 A1
(43) Pub. Date: Nov. 5, 2026

(54) ROBOTIC ARM DEPLOYMENT SYSTEM

(71) Applicant: Auris Health, Inc., Santa Clara, CA (US)

(72) Inventors: Nicholas J. EYRE, Sunnyvale, CA (US); Travis COVINGTON, Sunnyvale, CA (US); Alex C. SPIES, Redwood City, CA (US); Joseph L. DIAMOND, San Carlos, CA (US); Jingyi XU, Mountian View, CA (US); Kyle DHINDSA, Santa Clara, CA (US); Bryan James CULVER, Mountian View, CA (US); Abigail June LINDQUIST, Menlo Park, CA (US)

(21) Appl. No.: 19/716,019

(22) Filed: Jun. 22, 2026

Related U.S. Application Data
(63) Continuation of application No. PCT/IB2025/060456, filed on Oct. 14, 2025.
(60) Provisional application No. 63/707,183, filed on Oct. 14, 2024.

(51) Int. Cl. B25J 5/02; A61B 34/30; B25J 19/00

(52) U.S. Cl. CPC B25J 5/02; A61B 34/30; B25J 19/0004; B25J 19/0025

(57) ABSTRACT

A robotic system includes an arm support having a track. A cable path extends alongside the track, an arm carriage is movably mounted to the track, and a robotic arm is mounted to the arm carriage such that the robotic arm moves with the arm carriage along the track. A spool is rotatably mounted to the arm carriage such that the spool rotates relative to the arm carriage as the arm carriage moves along the track. An electrical cable has a first portion attached to the spool and a second portion attached to the cable path such that the electrical cable winds onto the spool from the cable path or pays out from the spool onto the cable path as the arm carriage moves along the track. A slip ring is mounted to the arm carriage, the slip ring electrically and rotatably coupling the electrical cable to the robotic arm.

### PRIORITY

This application is a continuation of International Application No, PCT/IB2025/060456, entitled “ROBOTIC ARM DEPLOYMENT SYSTEM,” filed Oct. 14, 2025, which claims the benefit of U.S. Patent Application No. 63/707,183, entitled “ROBOTIC ARM DEPLOYMENT SYSTEM,” filed Oct. 14, 2024, the disclosures of which are incorporated by reference herein.

### BACKGROUND

Minimally invasive medical procedures, such as laparoscopy, endoscopy, and robotically-assisted surgery, are increasingly used for the diagnosis or treatment of a variety of patient conditions. These techniques are attractive for their potential to minimize trauma to the patient, reduce recovery times, enhance surgeon precision, or facilitate new surgical approaches that may not be possible with traditional technologies. Such procedures can involve elongate instruments introduced through small incisions or natural orifices on a patient's body to reach an anatomical site. These instruments are then manipulated to observe or interact with target anatomy within the patient using the tips of these instruments. A surgeon may control these instruments while observing a real-time camera feed of the anatomical site.

### BRIEF DESCRIPTION OF DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts an example of a surgical system.

FIG. 2 depicts an example of a physician console from the surgical system of FIG. 1.

FIG. 3 depicts an example of the surgical system of FIG. 1 configured for combined endoscopic and laparoscopic surgery.

FIG. 4 depicts an example of an anatomical site from the surgical system of FIG. 3.

FIG. 5 depicts an example of a physician console for use with the surgical system of FIG. 3, configured for displaying endoscopic and laparoscopic views.

FIG. 6 depicts an example of a surgical robot for use with the surgical system of FIG. 3, configured for manipulating rigid and flexible instruments for combined endoscopic and laparoscopic surgery.

FIG. 7 depicts an example of a robotic manipulator for use with the surgical robot of FIG. 6, configured for manipulating a rigid instrument.

FIG. 8 depicts an example of a robotic manipulator for use with the surgical robot of FIG. 6, configured for manipulating a flexible instrument.

FIG. 9A depicts an example of a distal assembly for use with the robotic manipulators of FIGS. 7-8, with a tool driver and an instrument in a coupled configuration.

FIG. 9B depicts the distal assembly of FIG. 9A, with the tool driver and the instrument in a decoupled configuration.

FIG. 10 depicts an example of a deployment system for use with the surgical system of FIG. 1 or the surgical robot of FIG. 6.

FIG. 11A is a perspective view of the deployment system of FIG. 10 with portions hidden to illustrate internal or functional components of the system.

FIG. 11B is a bottom view of the deployment system of FIG. 11A.

FIG. 12A a top view of the deployment system of FIG. 11A corresponding to cross section A-A.

FIG. 12B a right side view of the deployment system of FIG. 11A corresponding to cross section B-B.

FIG. 12C a front view of the deployment system of FIG. 11A corresponding to cross section C-C.

FIG. 13A is a perspective view of the deployment system of FIG. 10 with some covers and internal components hidden to illustrate an assembly and operation of example of a cable winder of FIG. 12B.

FIG. 13B is a top view of the deployment system as seen in FIG. 13A.

FIG. 14A is a perspective view of the cable winder of FIG. 13A.

FIG. 14B is a right side view of the cable winder of FIG. 14A corresponding to cross section D-D.

FIG. 14C is a top view of the cable winder of FIG. 14B corresponding to cross section E-E of FIG. 14A.

FIG. 15A is a schematic diagram depicting multiple cable winders as seen in FIGS. 13A-14C in an example first state of operation.

FIG. 15B is a schematic diagram depicting the multiple cable winders of FIG. 15A in an example second state of operation.

FIG. 15C is a schematic diagram depicting the multiple cable winders of FIG. 15A in an example third state of operation.

FIG. 15D is a schematic diagram depicting the multiple cable winders of FIG. 15A in an example fourth state of operation.

FIG. 16A is a perspective view of an example of a carriage actuator of FIG. 12B with other portions of the deployment system hidden.

FIG. 16B is an enlarged view of the carriage actuator from the perspective view of FIG. 16A.

FIG. 16C is a top view of the carriage actuator from the enlarged view FIG. 16B.

FIG. 17A is a perspective view of an example of a brake of FIG. 12B with other portions of the deployment system hidden.

FIG. 17B is an enlarged view of the brake from the perspective view of FIG. 17A.

FIG. 17C is a top view of the brake from the enlarged view FIG. 17B.

FIG. 18A is an enlarged perspective view of an example variation of the brake of FIG. 17A.

FIG. 18B is a top view of the brake from the enlarged view FIG. 18A.

FIG. 19A is a perspective view of an example variation of the deployment system of FIG. 10.

FIG. 19B is a perspective view of the deployment system of FIG. 10 with cables attached.

FIG. 20A is a perspective view of an example configuration of the deployment system of FIG. 10, with robotic arms in a stowed configuration.

FIG. 20A is a perspective view of an example configuration of the deployment system of FIG. 10, with robotic arms in a deployed configuration.

FIG. 21A is a top view of an example configuration of the deployment system of FIG. 10, with robotic arms in a stowed configuration.

FIG. 21B is a top view of an example configuration of the deployment system of FIG. 10, with laparoscopic arms deployed in a 2 +2 configuration and an endoscopic arm positioned at a head of a surgical table.

FIG. 21C is a top view of an example configuration of the deployment system of FIG. 10, with laparoscopic arms deployed in a 3 +1 configuration and an endoscopic arm positioned at a foot of a surgical table.

FIG. 22A is a perspective view of an example configuration of the deployment system of FIG. 10 in use for an esophageal procedure.

FIG. 22B is a perspective view of an example configuration of the deployment system of FIG. 10 in use for a thoracic procedure.

FIG. 22C is a perspective view of an example configuration of the deployment system of FIG. 10 in use for a perineal procedure.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description and appended drawings contain certain examples and configurations of this technology and are not intended to be an exhaustive disclosure of the only configurations in which the technology may be practiced. Other examples, features, aspects, embodiments, and advantages of the technology will be apparent to those skill in the art from this disclosure. As will be realized, the technology described herein is capable of other different and obvious aspects, all without departing from the inventive concepts disclosed herein. Accordingly, the drawings and descriptions should be regarded as illustrative in nature and not restrictive. In some instances, well-known structures and components are not described in detail or are shown in block diagram form to avoid obscuring concepts of this technology.

Minimally invasive procedures such as laparoscopy and endoscopy may allow a physician to control elongate instruments introduced through small incisions or natural orifices on a patient's body to observe or interact with anatomical sites within the patient.

Laparoscopic procedures, for instance, can involve rigid surgical instruments introduced through one or more small incisions on a patient's abdomen. These laparoscopic instruments are manipulated from outside the patient's body through these small incisions to perform various surgical functions. The physician can perform surgical tasks, such as cutting, cauterizing, or grasping while observing a real-time camera feed of the internal anatomical site captured with one of these instruments (a rigid endoscope referred to as “laparoscope”). Because the physician views and interacts with organs or anatomical structures through small ports, rather than directly viewing such anatomical structures through a large incision, laparoscopic procedures are typically less invasive than open surgery.

Endoscopic procedures, for instance, can involve flexible instruments introduced through natural orifices on a patient, such as a mouth or perineal access point. Because these instruments are flexible, they can traverse through a lumen of a patient, which may follow a tortuous path, to reach a target anatomical site. The physician can visualize the anatomy using images obtained from a flexible endoscope to examine or diagnose conditions, or the physician may perform procedural tasks such as taking a sample or applying energy to the target site accessed with the flexible instrument. Because these techniques may involve accessing anatomy through natural orifices, they may be less invasive than laparoscopic techniques while allowing the physician to access or visualize regions of the patient anatomy that may not be readily reachable using laparoscopic techniques, such as the inside of organs like the stomach or colon.

Combining endoscopic and laparoscopic techniques can provide benefits of both approaches, with the flexibility for a physician to view and/or manipulate anatomy from endoscopic or laparoscopic perspectives. Among other things, technologies are described herein that facilitate endoscopic procedures, laparoscopic procedures, and robotic procedures. In some configurations, robotic systems and methods are described to facilitate combined use of endoscopic and laparoscopic approaches. In some configurations, a robotic system is provided that allows a single user to interact with a patient anatomy from endoscopic and laparoscopic approaches.

These and other features of the disclosed technology are further described below with respect to examples shown in the figures described below. It will be appreciated that there are various technical features and concepts disclosed herein which may be practiced independently from each other, in various combinations, or in other contexts beyond the particular examples shown and described with respect to these figures. Accordingly, these examples are explanatory in nature but should not be construed as limiting the scope of inventive subject matter to the precise examples disclosed.

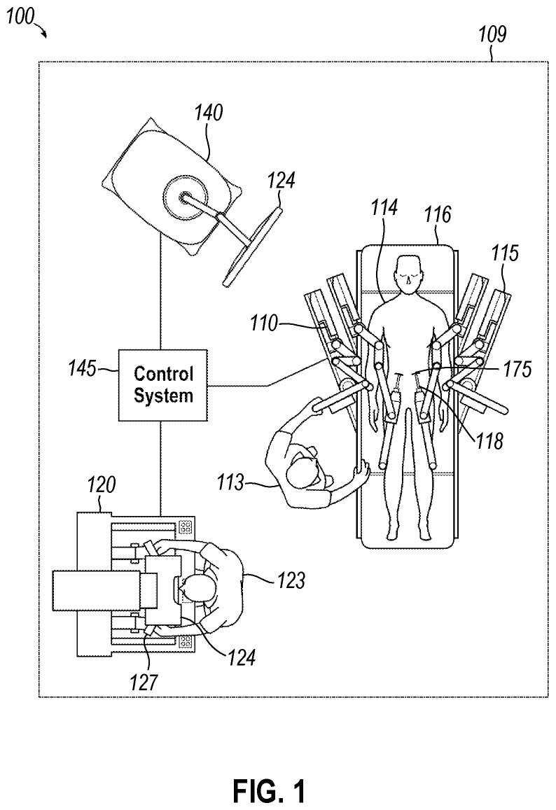

FIG. 1 depicts an example of a surgical system, in accordance with some embodiments. The surgical system 100 can be used to perform a variety of surgical procedures to diagnose and/or treat a patient 114. Examples of procedures include laparoscopy, endoscopy, thoracoscopy, urological procedures, and/or gastrointestinal (GI) procedures. As illustrated, the surgical system 100 is implemented as a robotic surgical system deployed for robotically-assisted procedures.

As seen in FIG. 1, surgical system 100 includes a surgical robot 110, a physician console 120, and a support tower 140. These components are set up in procedure area 109, such as an operating room or an endoscopy suite, and may be used in concert with each other to perform a procedure on patient 114. Components of the surgical system may be coupled physically, communicatively, and/or operatively as appropriate to facilitate operation of the surgical system 100. For instance, any two or more of the surgical robot 110, physician console 120, support tower 140, and/or other components of the surgical system may be interconnected via electrical signal lines, cabling, and/or wireless interconnections. In some configurations, components of the surgical system 100 can be situated in a common room or site. In some configurations, components of the surgical system 100 can be distributed across two or more rooms or sites that are remote from each other, where such components can be communicatively coupled over a network to implement a surgical procedure.

Surgical robot 110 is configured to interact with a patient 114 and perform various tasks. Surgical robot 110 can be actuated based on commands received from physician console 120. Such movements by the robot may be referred to as teleoperation (or telemanipulation), as they involve manipulations that are performed by the robot under human control, rather than fully autonomously. Alternatively, or in combination, surgical robot 110 can be configured to implement one or more tasks fully autonomously or semi-autonomously.

In the illustrated example, surgical robot 110 includes one or more robotic manipulators 115 configured to manipulate one or more instruments 118 (also referred to herein as “tools”). Examples of instruments include graspers, forceps, scissors, scopes, hooks, needle drivers, staplers, biopsy tools, energy delivery instruments, suction devices, irrigation devices, sheaths, snares, and various elongate instruments having flexible or rigid shafts that may be inserted into a patient's body. In some instances, an instrument may provide a combination of two or more functions to thereby provide two or more of these instrument types in a single device. Examples of such instruments include bipolar forceps (which handle tissue and deliver energy), and suction-irrigators (which provide both suction and irrigation of fluids). Surgical robot 110 can use distal portions of instruments 118 to interact with the patient or perform various procedure tasks, such as manipulating tissue or capturing endoscopic images. In various configurations, the robotic manipulator(s) 115 may be configured to manipulate multiple different types of instruments within a particular procedure and/or across different procedures, allowing the robot to use a variety of instruments to perform a variety of surgical functions. Each of the instruments 118 may be actuatable by the surgical robot 110 in one or more degrees of freedom (DOFs) of the instrument 118 (e.g., one or more DOFs where a tip or portion of the instrument moves relative to a handle or other portion of the instrument upon actuation). In some variations, any one or more of the instruments 118 may be non-actuated.

Instruments 118 can be inserted into a body of patient 114 through one or more ports to access an anatomical site within the patient's body. Ports may be positioned at one or more access points 175 provided by, for instance, laparoscopic incisions, cannulas, and/or natural orifices on a patient's body to provide an access channel for the instruments 118. An instrument 118 may be introduced into the patient's body through a port and advanced to the target anatomical site within the body. In some variations, procedures may involve one or several ports positioned at one or several access sites on the patient. In some variations, each port may be used to introduce one or multiple instruments concurrently or sequentially.

The robotic manipulators 115 can each include one or more actuators (e.g., motors) that can be electronically controlled to manipulate the instruments 118. For example, a robotic manipulator 115 can be actuated to control a position of an instrument 118 within the patient's body and/or to actuate mechanisms of the instrument (e.g., to articulate or operate an instrument tip in one or more degrees of freedom). In some variations, surgical robot 110 includes multiple robotic manipulators 115 configured to manipulate multiple instruments 118. For example, the surgical robot 110 can include two, three, four, five, six, or more robotic manipulators 115, where each manipulator manipulates one or more corresponding instruments 118.

Each robotic manipulator 115 can include, for example, a robotic arm having a series of links connected by a series of joints. A distal end of the robotic arm can be coupled with the corresponding instrument, and a proximal end of the robotic arm can be coupled with a support of the robot. Alternatively, or in combination, a robotic manipulator can include a carriage or motorized platform that may move along a track to control an instrument or interact with patient 114. In some instances, one or more users, such as one or more members of surgical staff 113, can mount or couple various instruments 118 to the various robotic manipulators 115 during initial set up and/or throughout a procedure to exchange instruments. As instruments are mounted to the various robotic manipulators 115, surgical robot 110 can be configured to detect presence and/or identify the corresponding instruments using sensing or identification technologies, such as optical sensing, magnetic sensing, radio frequency identification (RFID), or the like.

In the example shown in FIG. 1, surgical robot 110 is configured as a table-based system, where the robotic manipulators 115 are physically coupled to or integrated with a surgical table 116 (also referred to herein as an “operating table” or “patient support”), which supports patient 114. In some variations, the surgical robot 110 can be configured as a robotic cart that can be positioned beside the patient 114 and/or beside the surgical table 116. Alternatively, or in combination, the robot can be configured as a boom-based robot, where robotic manipulators descend from an overhead boom suspended above the patient. Such an overhead boom can be supported, for example, by a cart beside the patient or from a ceiling of an operating room. In some variations, the surgical robot 110 can include one or multiple robotic carts, where each cart supports one or multiple robotic manipulators or robotic arms, and where the multiple robotic carts are configured to operate in concert with each other. For example, in some variations, a distributed or modular surgical robot can involve a modular cart system, where multiple carts are positioned beside the surgical table 116 and each cart supports a robotic manipulator 115 that manipulates a corresponding instrument 118.

Physician console 120 can be configured to provide inputs or receive outputs to or from the robot 110 or the instruments 118. As illustrated, physician console 120 includes one or more input devices 127, which a user (e.g., physician 123) can operate to provide commands for teleoperation of the robot 110. Input device 127 can include, for example, a handheld device that the physician 123 can manipulate with one or more hands to provide input to the system. In some variations, the physician console 120 can employ one or several types of input devices to provide various modes for the physician 123 to interact with the surgical system 100. Examples of input devices include pendants, gimbal-based controllers, graspers, touch sensors, trackballs, joysticks, buttons, and/or foot pedals.

Surgical system 100 can also include one or more displays 124, which can be configured to present images for observation by physician 123. For example, physician console 120 can include display 124 can be configured to display a scope view derived from endoscopic images (e.g., a video feed) captured by an instrument 118. This can facilitate control of the surgical robot 110 by the physician 123 via the input device(s) 127, while the physician views a real-time camera feed of the anatomical site within the patient's body. Alternatively, or in combination, the display(s) 124 can be configured to display supplemental information associated with the surgical system or procedure, such as, for example, pre-operative images, navigation information, interactive menus, and/or status information associated with the instruments, the robot, and/or the surgical system. Examples of displays that may be employed by surgical system include flat panel displays, stereoscopic displays, head-mounted displays, liquid crystal displays (LCD), organic light emitting diode (OLED) displays, touch screen displays, and/or various other types of electronic display devices.

The support tower 140 can interact with surgical robot 110, instruments 118, and/or physician console 120 to provide various supporting functionality to the system, such as vision processing, fluidics, and/or energy generation. For example, the support tower 140 can process images received from an endoscope, generate light to an endoscope to illuminate the surgical site, provide suction and/or irrigation from the surgical site, operate localization sensors, such as shape sensors and/or electromagnetic (EM) sensors, and/or generate energy provided to one or more of the instruments 118 (e.g., for electrosurgery functions such as coagulating or cutting tissue). Alternatively, or in combination, support tower 140 can provide an interface for one or more users, such as surgical staff 113, to interact with the surgical system (e.g., provide inputs to the surgical system and/or observe outputs of the surgical system). In the illustrated example, support tower 140 includes one or more tower displays 124 that can be configured to present any of the same information described herein with respect to the physician console and/or additional information.

In the illustrated example, surgical robot 110, physician console 120, and support tower 140 are illustrated as separate components that may be positioned in various locations in procedure area 109. In some variations, any two or more of these components may be integral. For example, in some configurations, the support tower 140 may be provided as an integral component of the physician console 120 or surgical robot 110.

Control system 145 can be communicatively coupled to robot 110, physician console 120, and/or support tower 140. Control system 145 includes processing circuitry and memory configured to implement functions of surgical system 100, such as controlling or actuating robot 110, controlling or operating the instruments, or processing inputs or outputs to or from physician console 120. For example, processing circuitry of the control system 145 can be configured via hardware or software programming to implement any functions described further herein in connection with operation of surgical system 100, including carrying out any of the methods described herein. Examples of processing circuitry include one or more central processing units (CPUs), graphics processing units (GPUs), field programmable gate arrays (FPGAs), application specific integrated circuits (ASICs), or other processors configured to process inputs or outputs for the surgical system 100. As used herein, the term “processor” can encompass a single processing chip or integrated circuit, or multiple processing chips or integrated circuits that may be co-located or distributed in different locations and configured to execute functions described herein. Memory can store instructions that, when executed by the processor, cause the surgical system to perform any of the methods or functions described herein. As used herein, the term “memory” can encompass any suitable non-transitory processor-readable medium embodied in one or several memory devices, such as hard drives, flash memory, solid state memory, storage discs, or tapes. Components of the control system 145 may be physically located in, or physically connected to, components of the surgical system 100, such as the robot 110, the physician console 120, and/or the support tower 140. Alternatively, or in combination, components of control system 145 may be communicatively coupled to components of surgical system 100 via various wired or wireless interconnections.

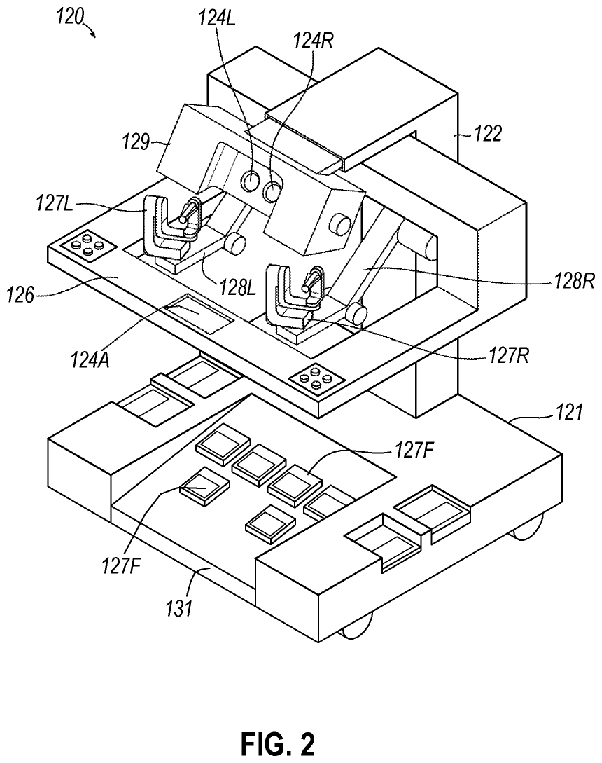

FIG. 2 depicts an example of physician console 120, in accordance with some embodiments. The physician console 120 can, for instance, be incorporated in surgical system 100 seen in FIG. 1. As noted above, physician console 120 (sometimes referred to herein as a “surgeon console”) can provide an interface for a physician (e.g., a surgeon) to interact with the surgical system 100. For example, a physician may interact with the physician console 120 to control the surgical robot and/or observe images of a surgical site.

As seen in FIG. 2, physician console 120 can include a console base 121, a pillar 122 (also referred to as a “column”) coupled to the console base 121, and a viewer assembly 129 coupled to the pillar 122. Physician console 120 further includes an armrest 126, which is coupled to and supported by pillar 122. Viewer assembly 129 can be supported by the console base 121 via the pillar and can provide a primary display for a physician to view endoscopic images of the anatomical site. In some configurations, viewer assembly 129 is configured to display anatomical images obtained from both flexible and rigid laparoscopes, as further described herein. In the illustrated example, viewer assembly 129 houses an immersive, three-dimensional, stereoscopic display, which includes a left eye display 124L and a right eye display 124R. This immersive display can present three-dimensional images to the physician when the physician inserts their head into the viewer housing. In some variations, the physician console 120 may be provided with an open design, where the viewer assembly 129 provides a two-dimensional or three-dimensional flat panel display that can present images to the physician without a need for the physician to insert their head into a viewer housing. In some variations, viewer assembly 129 may be provided by a wearable headset (e.g., a head-mounted display).

In the example shown in FIG. 2, physician console 120 also includes a pair of input devices including a left hand input device (HID) 127L and a right HID 127R, configured to be manipulated by the physician's left and right hands, respectively (an HID is also sometimes referred to herein as a “human interface device”). Each of the HIDs can include a handle and/or finger inputs that are manipulated by a user's hands to control a corresponding instrument and/or corresponding robotic manipulator. For example, the left HID 127L may be controlled by a user's left hand to control a left-hand instrument manipulated by a first robotic arm of the surgical robot, and the right HID 127R may be controlled by a user's right hand to control a right-hand instrument manipulated by a second robotic arm of the surgical robot. In the illustrated example, each of the HIDs is physically supported by an armrest 126 of the physician console 120 and/or the pillar 122 by a respective positioning arm, including a left positioning arm 128L and a right positioning arm 128R. Such positioning arms can include a series of links and series of joints, including a gimbal-based support, that supports the respective HID in space while permitting the respective HID to be manipulated in six degrees of freedom to control a corresponding position (e.g., location and/or orientation) of the respective instrument. Alternatively, or in combination, each of the left HID or right HID can include graspers and/or buttons that may be actuated by the user's respective hands to actuate the instrument (e.g., to open or close instrument jaws) or control other functions of the surgical system. The illustrated configuration depicts grounded HIDs that are physically grounded to the console via positioning arms. In some variations, the physician console can employ ungrounded HIDs, such as free-floating and/or wireless input devices.

As seen in FIG. 2, physician console 120 can also include a foot pedal assembly 131 (e.g., a footboard) having one or more foot pedals 127F. The foot pedal(s) 127F may be coupled to or otherwise positioned at the base 121 of the physician console and may be actuated by a user's feet to control various functions of the system. For example, in some configurations, various foot pedals may be used to perform ancillary functions of the system, such as activating energy delivery, switching control of instruments, clutching instruments, firing a staple, or toggling a menu.

In some variations, the physician console 120 can include one or more additional or secondary displays. In the illustrated example, physician console 120 includes an auxiliary display 124A, which can be implemented as a touchscreen display positioned on the armrest 126. The auxiliary display 124A can provide an additional interface for a physician to interact with the system. For example, the auxiliary display 124A can provide an additional output interface for displaying various settings or status information associated with the surgical system 100. Alternatively, or in combination, the auxiliary display 124A can provide an input interface for controlling system settings.

In some variations, it may be beneficial for a surgical system to facilitate hybrid approaches or techniques, such as the use of both flexible and rigid instrumentation and/or access to internal anatomical sites via different types of entry points. For instance, a combined endoscopic and laparoscopic system may allow for complimentary hybrid techniques that combine benefits of both laparoscopy and endoscopy.

An example of a patient condition that may benefit from combined endoscopy and laparoscopic surgery is the diagnosis or treatment of colorectal polyps. Flexible endoscopic instrumentation may access the inside of a colon through a perineal access point (e.g., anus), then be advanced to a target polyp for further examination or treatment. If the polyp is benign or can be treated without a need for surgical resection, then the procedure may proceed using a purely endoscopic technique. In cases where further intervention is needed, the procedure may then escalate to laparoscopic intervention, where rigid instruments can perform surgical manipulation and/or resection of the malignant tissue. Alternatively, or in combination, such procedures may involve concomitant use of endoscopic and laparoscopic instrumentation to target the polyp or target site, for instance, to provide laparoscopic assistance to endoscopic examination or treatment of the polyp, and/or to provide endoscopic assistance to laparoscopic examination or treatment. Such hybrid techniques may benefit from the concomitant use of instruments for viewing or manipulating the same target anatomy from different perspectives, thereby facilitating enhanced surgical techniques or improved visualization of the target site.

Various types of patient conditions and procedures may benefit from hybrid techniques or combined endoscopic and laparoscopic surgery, providing potential for improved outcomes such as reduced complications, broadened use of organ preserving technique, reduced risk of injury, and reduced length of hospital stays. Examples of procedures that may benefit from combined endoscopic and laparoscopic surgery include upper GI procedures, lower GI procedures, and thoracic procedures.

Upper GI procedures can, for instance, involve one or more flexible instruments (such as an endoscope and/or working channel tools) introduced to an anatomical site (such as the stomach or upper GI tract) through a patient's mouth. With a combined endoscopic and laparoscopic system, one or more rigid instruments, such as a laparoscope and/or one or more other laparoscopic instruments, may access the anatomical site through one or more incisions on the patient's abdomen to access the same anatomical site from a different perspective (for example, from outside the stomach or upper GI tract).

Lower GI procedures can, for instance, involve one or more flexible instruments introduced to an anatomical site (such as the colon or lower GI tract) through a perineal access point. With a combined endoscopic and laparoscopic system, one or more rigid instruments may access the anatomical site through one or more incisions on the patient's abdomen to access the same anatomical site from a different perspective (for example, from outside the colon or lower GI tract).

Thoracic procedures can, for instance, involve one or more flexible instruments introduced to an anatomical site (such as the lung or pulmonary region) through a patient's mouth. With a combined endoscopic and laparoscopic system, one or more rigid instruments may access the anatomical site through one or more incisions on the patient's chest to access the same anatomical site from a different perspective (for example, from outside the lung or pulmonary region).

Robotic systems may facilitate these and other hybrid approaches, providing an integrated robotic platform that allows one or more users to control and/or visualize anatomy using endoscopic and laparoscopic approaches. In some variations, a single user may be able to control both endoscopic and instrumentation using a robotic surgical system.

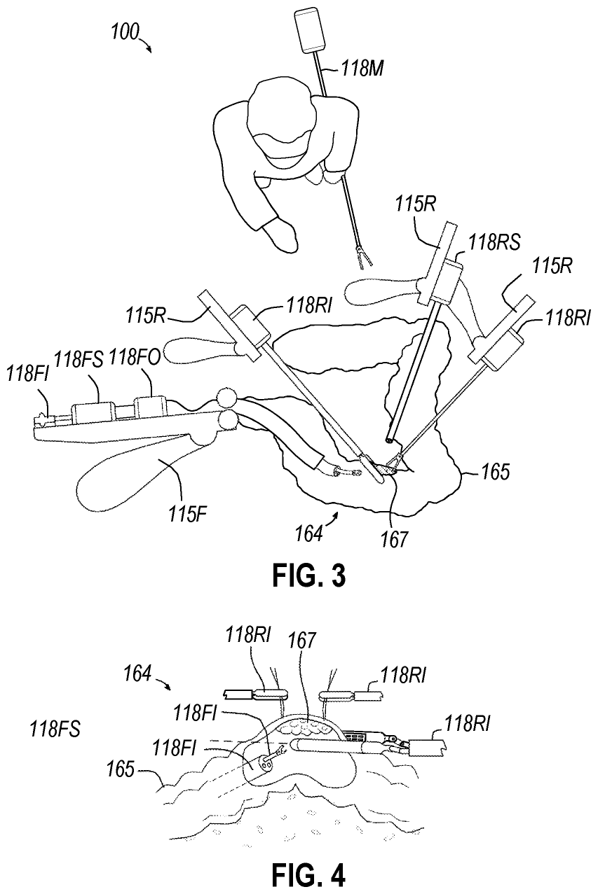

FIG. 3 depicts an example configuration of surgical system 100 in use for combined endoscopic and laparoscopic surgery, in accordance with some embodiments.

As seen in FIG. 3, surgical system 100 employs multiple robotic manipulators 115 and multiple instruments 118 interacting with an anatomical site 164. As seen in FIG. 3, anatomical site 164 encompasses an organ 165 (a colon in this example), where instruments operate on a target structure 167 within the organ (a polyp in this example) using both endoscopic (e.g., endoluminal) intervention and laparoscopic surgery.

In the illustrated example, three robotic manipulators 115R (also referred to herein as “rigid instrument manipulators” or “laparoscopic manipulators”) control three corresponding rigid instruments 118R from a laparoscopic approach, including a rigid scope 118RS (e.g., a laparoscope) for capturing images of the anatomical site, and a pair of surgical instruments 118RI (e.g., rigid laparoscopic instruments) for manipulating tissue. Rigid scope 118RS can include a camera for capturing images of the anatomical site 164 while the surgical instruments 118RI perform various tasks or manipulations on the anatomical site 164. For instance, rigid scope 118RS may include one or more image sensors arranged at its distal tip, along with corresponding optics, and a light source or light pipe at the distal tip for illuminating the anatomical site 164 while the surgical instruments 118RI operate within the field of view (FOV) of the rigid scope 118RS. In some variations, rigid scope 118RS includes a stereoscopic camera to provide three-dimensional (3D) images. In some variations, rigid scope 118RS includes a monoscopic (or “monocular”) camera to provide two-dimensional (2D) images. Each surgical instrument 118RI can include an end effector adapted for one or more tasks, such as grasping, sealing, and/or cutting, and the end effector may optionally be articulatable or actuatable by the corresponding rigid instrument manipulator 115R.

In the illustrated example, a robotic manipulator 115F (also referred to herein as a “flexible instrument manipulator” or “endoscopic manipulator”) controls three flexible instruments 118F. Here, the flexible instruments 118F are mounted to the same flexible instrument manipulator 115F in a coaxial or telescoping arrangement, and these instruments interact with the anatomical site from an endoluminal approach. The flexible instruments include an overtube 118FO, a flexible scope 118FS (e.g., a colonoscope) extending into a channel of overtube 118FO, and a working channel instrument 118FI extending into a channel of the flexible scope 118FS. Flexible scope 118FS may be robotically steerable or controllable through the lumen (colon) to reach the target structure 167. Overtube 118FO may also be robotically steerable or controllable to help support or guide the flexible scope 118FS as it navigates through the lumen (colon). Working channel instrument 118FI may be introduced through a working channel of the flexible scope 118FS to interact with the target structure 167. For example, working channel instrument 118FI may be manipulated to remove tissue, apply energy, or deliver therapeutics. Flexible scope 118FS can include a camera for capturing images of the anatomical site 164 while the instruments perform various tasks or manipulations on the anatomical site 164. For instance, flexible scope 118FS may include one or more image sensors arranged at its distal tip, along with corresponding optics, and a light source or light pipe at the distal tip for illuminating the anatomical site 164. In some variations, flexible scope 118FS includes a stereoscopic camera to provide three-dimensional (3D) images. In some variations, flexible scope 118FS includes a monoscopic camera to provide two-dimensional (2D) images. In some variations, the rigid scope 118RS includes a stereoscopic (3D) camera while flexible scope 118FS includes a monoscopic (2D) camera, thereby providing enhanced visualization for precise 3D control from the laparoscopic view, while reducing cost or complexity for the flexible endoluminal camera.

In some variations, one or more manual instruments 118M may be utilized in concert with the surgical system 100. For example, a manual laparoscopic instrument may be manipulated by a beside user through a laparoscopic port. Alternatively, or in combination, one or more of the illustrated instruments may be configured for manual control, such as the working channel instrument 118FI or flexible scope 118FS.

FIG. 4 depicts an enlarged view of the anatomical site 164, to further illustrate how endoluminal and laparoscopic instruments may be used in tandem to interact with the operative site. Here, the distal tips of three surgical instruments 118RI are shown manipulating the tissue (e.g., organ 165), while rigid scope 118RS from FIG. 3 is not visible in this enlarged view. The distal tips of flexible endoscope 118FS and working channel instrument 118FI are shown observing or manipulating the target structure 167, while overtube 118FO from FIG. 3 is not visible in this enlarged view. As illustrated, the endoluminal and laparoscopic instruments may interact with the anatomical site 164 in concert with each other from different approaches, such as opposing sides of the organ wall. Here, the flexible instruments 118F observe or interact with tissue from within the organ 165, while rigid instruments 118R interact with the target from outside the organ 165. Using these instruments in concert may allow various advanced techniques, such as, for example, grasping or positioning tissue with the laparoscopic instruments to facilitate visualization of the site with the endoluminal instruments.

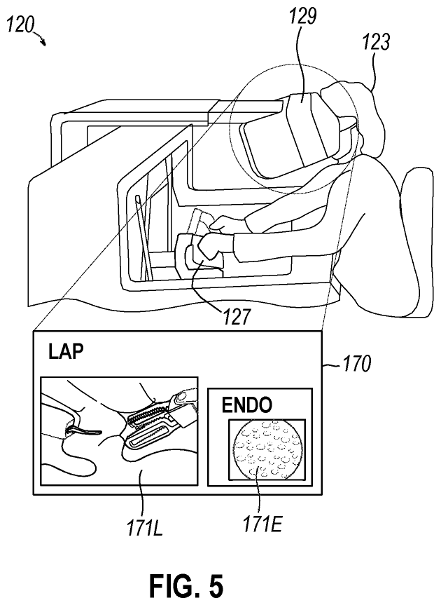

FIG. 5 depicts an example variation of physician console 120 configured for combined endoscopic and laparoscopic surgery, in accordance with some embodiments. Physician console 120 may be used, for instance, in connection with the surgical system 100 as seen in FIGS. 3-4.

As illustrated, physician console 120 may present a graphical interface 170 (also referred to herein as a “display interface”) on or more of the displays 124 (e.g., on viewer assembly 129) for viewing by physician 123. Graphical interface 170 may display one or more scope views or images of the anatomical site obtained from one or more scopes of the system. In the illustrated example, graphical interface 170 is configured to display both a laparoscopic view 171L and an endoluminal view 171E. Laparoscopic view 171L can, for instance, include one or more images obtained from rigid scope 118RS (FIG. 3). Endoluminal view 171E can, for instance, include one or more images obtained from flexible scope 118FS (FIG. 3). Physician console 120 is configured to receive input at one or more input devices 127, including commands for controlling any one or more of the instruments while the physician observes the graphical interface 170 presented on the viewer.

In the illustrated example, graphical interface 170 presents both the laparoscopic view 171L and the endoluminal view 171E concurrently (simultaneously), which may allow physician to concurrently view the anatomical site captured by the scopes from different perspectives. The concurrently displayed views may be presented, for instance, with a side-by-side configuration or a picture-in-picture configuration. In some variations, one of the views may be displayed larger than the other view or located more centrally within the display interface relative to the other view to be presented as a primary view, while the other of the views may be displayed smaller than the other view or less centrally than the other view to be presented as a secondary view. The physician console 120 may allow the user to select or switch between the views by providing input to one or more input devices, such as, for instance, one or more of the HIDs 127L, 127R, one or more of the foot pedals 127F, and/or a touch interface of the auxiliary display 124A (FIG. 2). In some variations, the views are displayed asynchronously, or not at the same time. In such variations, physician console 120 may be configured to receive input from physician 123 at any one or more of the input devices to switch between the views or select which of the views to be presented on the display.

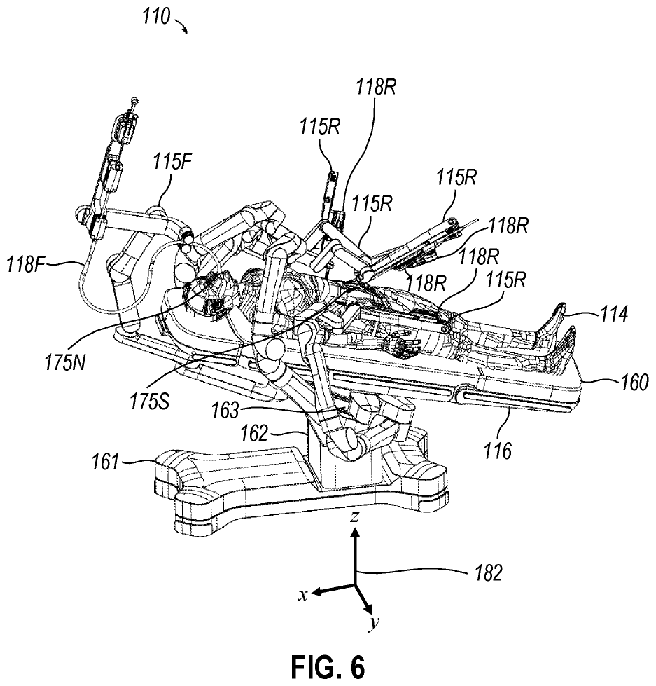

FIG. 6 depicts an example variation of surgical robot 110 configured for combined endoscopic and laparoscopic surgery, in accordance with some embodiments. Surgical robot 110 may be used, for instance, in connection with surgical system 100 as seen in FIGS. 3-4. Surgical robot 110 may be controlled, for instance, based on commands received from physician console 120 as seen in FIG. 5.

As seen in FIG. 6, surgical robot 110 includes multiple robotic manipulators 115R, 115F coupled to operating table 116 (also referred to herein as a “surgical table”). Operating table 116 includes a table base 161, a column 162 extending vertically from the table base 161, and a table top 160 supported by the column 162. Patient 114 can be supported by the operating table 116 on the table top 160, which can be movable or actuatable relative to the table base 161 and/or column 162 to adjust positioning of the patient 114 supported thereon.

The table top 160 can be adjustable to various positions, angles, or orientations relative to the table base 161 to provide a desired positioning of the patient for a given procedure or surgical task. The table top 160 can have one or more DOFs relative to table base 161, where such DOFs can be actuated by motorized mechanisms within the surgical table (e.g., one or more motors within column 162 and/or table base 161). FIG. 6 depicts a coordinate system 182 that can be used to define the DOFs of the table top 160 and/or surgical robot 110. Here, the coordinate system 182 is defined in cartesian coordinates relative table base 161, which provides a base frame of reference that can also be aligned relative to a gravitational frame of reference of floor of procedure area 109 (FIG. 1) as the table base 161 rests on the floor. Table top 160 can have, for instance, a height DOF (also referred to as Z-height or table top lift), where the table top 160 can be raised or lowered relative to table base 161 (e.g., translation along Z-axis of coordinate system 182). Table top 160 can have a Trendelenburg DOF, where the table top 160 can be rotated about y-axis relative to table base 161 to raise or lower the foot of the table relative to the head of the table (e.g., to place the table top 160 and patient 114 in a reverse Trendelenburg position, as seen in FIG. 6, where the head of the table top 160 and patient 114 is raised relative to the foot of the table top 160 and patient 114, or to place the table top in a Trendelenburg position, where the head of the table top 160 and patient 114 is lowered relative to the foot of the table top 160 and patient 114). Table top 160 can have a tilt DOF (also referred to as “lateral tilt”), where the table top 160 can be rotated about x-axis relative to table base 161 to raise or lower the left side the table relative to the right side of the table. In some variations, table top can include more or fewer degrees of freedom than those described above. For instance, table top 160 can include any one or more DOFs associated with movement of the table top 160 relative to the table base 161. For instance, table top 160 can have any one or more of six DOFs, which include three DOFs associated with translational movement along the x, y, or z axes, respectively, and three DOFs associated with rotational movement about the x, y, or z axes, respectively.

Robotic manipulators 115 (115R, 115F) are mounted to an arm support 163, which can be coupled to the operating table 116 to provide a support base for each of the robotic manipulators 115. In the illustrated example, arm support 163 is coupled to operating table 116 distal to the Z-height, Trendelenburg, and tilt DOFs of the table top 160 such that the arm support 163, and therefore the robotic manipulators 115 (115R, 115F), move in concert with each other with unified motion as the table top 160 is actuated on those DOFs relative to the table base 161. In some variations, arm support 163 may be fixed relative to the table top 160, such that the arm support 163 and the table top 160 move in concert with each other with unified motion as the table top 160 is actuated in all of its DOFs relative to the table base 161. In some variations, arm support 163 is coupled to the column 162, the table base 161, or a cart separate from the operating table 116, in which case the arm support 163 may be movable or actuatable in any DOF independently from the table top 160. In the illustrated example, arm support 163 is positioned underneath the table top 160, such that the robotic manipulators 115 can extend around edges of the table top 160 when in a deployed configuration to position the instruments 118 in the workspace above or near the patient 114. In some variations, the robotic manipulators 115 may be movable to a stowed configuration, where the robotic manipulators are positioned beneath the table top 160 for stowage.

Robotic manipulators 115 can manipulate instruments 118 (118R, 118F) that are introduced into the patient through access points 175 (175N, 175S). In the illustrated example, surgical robot 110 includes four rigid instrument manipulators 115R that hold and manipulate four corresponding rigid instruments 118R. Each of the rigid instruments 118R may be introduced into the patient through a surgical incision 175S (e.g., a minimally invasive or laparoscopic incision). In some variations, each of the multiple rigid instruments 118R is introduced through its own respective port corresponding to its own respective incision. In some variations, two or more of the rigid instruments 118R are introduced through the same port corresponding to the same incision (e.g., for a single port surgical approach). In the illustrated example, surgical robot 110 also includes a flexible instrument manipulator 115F configured to manipulate a flexible instrument, which has a flexible shaft that may be introduced into the patient through a natural orifice 175N (e.g., a mouth). In some variations, the robotic manipulators 115 may be movable to various poses relative to the table top 160 to position the manipulators as appropriate for various procedures.

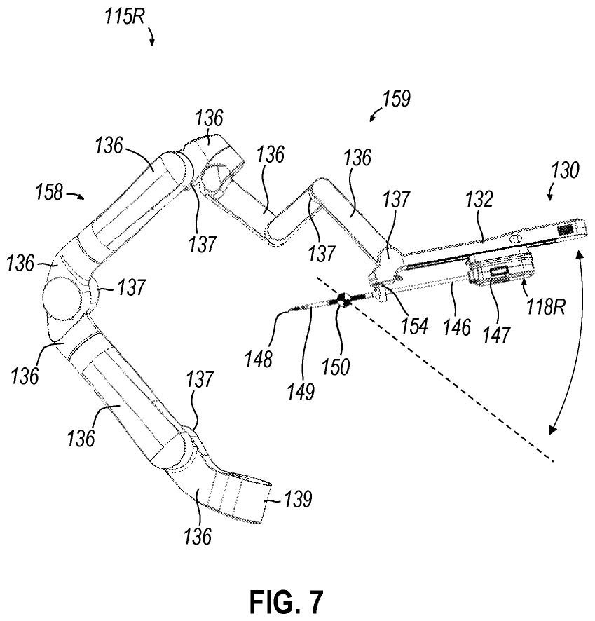

FIG. 7 depicts an example variation of rigid instrument manipulator 115R for controlling a rigid instrument 118R, in accordance with some embodiments. Rigid instrument manipulator 115R may be used, for instance, in connection with surgical robot 110 as seen in FIG. 6 and/or surgical system 100 as seen in FIG. 3.

As seen in FIG. 7, rigid instrument manipulator 115R includes a robotic arm having multiple links 136 connected by multiple joints 137. The links 136 may be arranged as a series of rigid bodies connected by joints 137 to form a kinematic chain that terminates with rigid instrument 118R. The robotic arm may include various types joints, such as one or more pitch joints, roll joints, and/or prismatic joints, each of which may constrain movement of its adjacent links around or along certain axes relative to others. Each joint 137 may include or be coupled to an actuator (e.g., a motor), which may be actuated to control movement of adjacent links relative to one another. Each joint 137 can include or be coupled to an encoder, which can measure position information associated with the joint (e.g., a joint angle), to provide robot kinematic data. A proximal end of the robotic arm may include an arm base 139, which may be coupled to, and supported by, a mounting structure of the surgical robot, such as arm support 163 (FIG. 6). Actuation of various joints of the robotic arm can move the distal end of the robotic arm to thereby control a position of the rigid instrument 118R in space, and to move the rigid instrument 118R relative to arm base 139.

A distal assembly 130 is arranged at the distal portion of the robotic arm. Distal assembly 130 includes a tool driver 132 (also referred to herein as an “instrument driver”) arranged at the distal end of the arm. Tool driver 132 is coupled to and supports rigid instrument 118R. Tool driver 132 is also coupled to and supports cannula 149, which is configured to receive and guide rigid instrument 118R.

The rigid instrument 118R includes an elongate shaft 146 and an instrument tip 148 arranged at a distal end of the elongate shaft 146. The elongate shaft 146 may rigidly support the instrument tip 148, which can provide an end effector for interacting with the anatomical site within the patient. Instrument shaft 146 can extend from the instrument base 147, which may provide a housing that contains mechanisms actuated by the tool driver 132 for actuating portions of the rigid instrument 118R. In some variations, the instrument tip 148 includes a robotic wrist and jaws at the distal end of the tool, which can be actuated to manipulate tissue or perform surgical tasks. In some variations, the rigid instrument 118R is non-actuated, such as some variations of a rigid laparoscope. The plurality of the joints of the robotic arm can be actuated to position and orient the tool holder 132, thereby positioning and orienting the cannula 149 and/or the instrument tip 148.

A cannula interface 154 provides a cannula holding portion of the tool driver 132. Cannula interface 154 is configured to engage the cannula 149 via, for example, a clamp, latch, or mechanical attachment, to hold and stabilize the cannula 149 with respect to the tool driver and with respect to the rigid instrument 118R mounted to the tool driver. The tool driver can include a carriage that moves along an elongate track to thereby advance or retract the instrument shaft 146 through the cannula 149. The tool driver 132 may be arranged at the distal end of a robotic arm such that articulation of the robotic arm positions and/or orients the tool driver 132 in space, thereby orienting the rigid instrument 118R and/or cannula 149.

FIG. 7 depicts an example of the rigid instrument manipulator 115R adapted for a rigid surgical instrument (e.g., a laparoscopic instrument). As seen in FIG. 7, rigid instrument manipulator 115R can manipulate or move rigid instrument 118R through a cannula 149 about a remote center of motion (RCM) 150, for example, by pivoting the rigid instrument 118R about RCM 150 in the direction of the arrow. As the elongate shaft 146 may rigidly support the instrument tip 148, moving or pivoting the instrument base 147 can cause a corresponding movement or pivoting of the instrument tip 148. The cannula 149 may, for example, provide a port that can be positioned at a small opening or incision on a patient's body, such as incision 175S (FIG. 6), to facilitate introduction of the rigid instrument 118R to an internal anatomical site. By maintaining the position of the RCM 150 during movements of the rigid instrument 118R and/or cannula 149, the robotic manipulator can control a position of the instrument tip 148 of the rigid instrument 118R while avoiding undue trauma or stresses to the patient's body wall as that rigid instrument 118R is moved or manipulated.

In some variations, the plurality of links 136 and joints 137 of the robotic arm can be divided into two segments. The first arm segment 158 includes a proximal set of the links 136 and joints 137, and may be referred to in some variations as a setup arm because it can position and adjust the RCM in space relative to the mounting fixture. The second arm segment 159 can include a distal set of the links 136 and joints 137, and may be referred to in some variations as the spherical arm because it can move the surgical instrument within a generally spherical workspace. The second arm segment 159 (e.g., spherical arm) can include a mechanism that mechanically constrains movement of the tool driver 132 around RCM 150, and accordingly, constrains movement of the tools mounted to the tool driver 132 (including rigid instrument 118R and cannula 149), around RCM 150. Such a mechanism may be referred to as a mechanical RCM or mechanical-based RCM. For instance, a first one of the links 136 and a second one of the links 136 of the spherical arm can be operatively coupled with a pulley mechanism to form a parallelogram that mechanically constrains movement about the RCM. The spherical arm can, for instance, have at least two degrees of freedom (DOFs). The first arm segment 158 (e.g., setup arm) can, for instance, have at least five DOFs provided by five of the joints 137 in the first segment. The proximal end of the first arm segment 158 can be mounted to an arm support (e.g., arm support 163 as seen in FIG. 6), while the distal end is coupled to the second arm segment 159.

In some variations, the robotic arm constrains motion about the RCM 150 via software or algorithms, rather than mechanical mechanisms. Such a configuration may be referred to as a software RCM or software-based RCM. In some variations, the robotic arm contains more or fewer joints and links, thereby providing more or fewer degrees of freedom for controlling motion of the robotic arm.

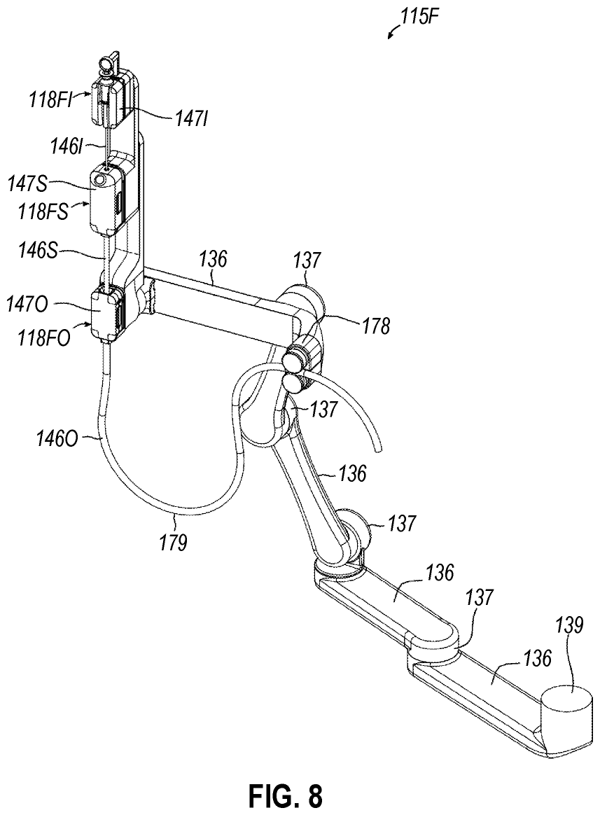

FIG. 8 depicts an example variation of flexible instrument manipulator 115F for controlling one or more flexible instruments, in accordance with some embodiments. Flexible instrument manipulator 115F may be used, for instance, in connection with surgical robot 110 as seen in FIG. 6 and/or surgical system 100 as seen in FIG. 3. Although flexible instrument manipulator 115F may share features in common with rigid instrument manipulator 115R, here the flexible instrument manipulator 115F as seen in FIG. 8 has a different architecture adapted to manipulate flexible instruments rather than rigid instruments. In some variations, flexible and rigid instruments can be manipulated by robotic manipulators having the same architecture as each other, and/or different architectures from those illustrated here.

As seen in FIG. 8, flexible instrument manipulator 115F includes a robotic arm having multiple links 136 connected by multiple joints 137. The links 136 may be arranged as a series of rigid bodies connected by joints 137 to form a kinematic chain that terminates with distal assembly 130. A proximal end of the robotic arm may include an arm base 139, which may be coupled to, and supported by, a mounting structure of the surgical robot, such as support 163 (FIG. 6). For example, actuation of various joints of the robotic arm can move the distal end of the robotic arm to thereby control a position of the rigid instrument 118R in space, and to move the rigid instrument 118R relative to arm base 139.

A distal assembly 130 is arranged at the distal portion of the robotic arm. Distal assembly 130 includes a tool driver 132 (also referred to herein as an “instrument driver”) arranged at the distal end of the arm. In this example, tool driver 132 is coupled to and supports multiple flexible instruments, which include flexible endoscope 118FS, working channel instrument 118FI, and overtube 118FO. These flexible instruments may be arranged in a coaxial or telescoping arrangement, and the tool driver 132 may include multiple instrument carriages arranged along a track to support the instrument bases of the respective instruments (e.g., working channel instrument base 147I, e.g., scope base 147S, and overtube base 147O). Each of the flexible instruments 118F includes a flexible shaft (146S, 146O, 146I) that may extend from its corresponding instrument base. Here, working channel instrument shaft 146I extends through the scope base 147S and into a working channel of the scope shaft 146S. Scope shaft 146S extends through the overtube base 147O and into a channel of the overtube shaft 146O. In some variations, the tool driver 132 may support more or fewer flexible instruments and/or different types of flexible instruments. For instance, the tool driver 132 may support one, two, three, or four flexible instruments in some variations. Each instrument base may provide a housing that contains mechanisms actuated by the tool driver 132 for actuating portions of the corresponding flexible instrument, for instance, to articulate, steer, or actuate the tip of the corresponding instrument. The plurality of the joints of the robotic arm can be actuated to position and orient the tool driver 132, thereby positioning and orienting the flexible instruments.

FIG. 8 depicts an example of the flexible instrument manipulator 115F adapted for flexible instruments (e.g., endoluminal instruments). As seen in FIG. 8, tool driver 132 can be coupled to and support feedroller assembly 178, which is configured to receive one or more of the flexible instrument shafts. Feedroller assembly 178 can include one or more feedroller wheels (e.g., a pair of opposing rollers) that can engage with the instrument shaft(s) received therein, and can be driven to advance or retract the shaft. In the illustrated example, the flexible instruments shafts are shown with a service loop portion 179, with the outermost overtube shaft 146O visible, though it will be appreciated that the scope shaft 146S and working channel tool shaft 146I can be housed within the overtube shaft 146O. The service loop portion 179 can provide slack in the instrument shaft(s). For instance, flexible instrument manipulator 115F can drive the feedroller assembly 178 in a first direction (distal or forward) to take in the service loop portion 179 and advance the shaft(s), or drive the feedroller assembly 178 in a second, opposite direction (proximal or backward) to let out the service loop portion 179 and retract the shaft(s).

In some variations, the service loop portion and/or feedroller assembly 178 is omitted, in which case the flexible instrument manipulator 115F can be configured to advance or retract the instrument shaft(s) solely via motion of the manipulator itself (e.g., articulation of the arm joints and/or movement of the instrument carriages of the tool driver 132). In some variations, an additional port, such as an introducer, may be coupled to or positioned proximate to the distal assembly 130 to facilitate introducing the instrument shafts into the anatomical opening of the patient. In some variations, the robotic arm contains more or fewer joints and links, thereby providing more or fewer degrees of freedom for controlling motion of the robotic arm.

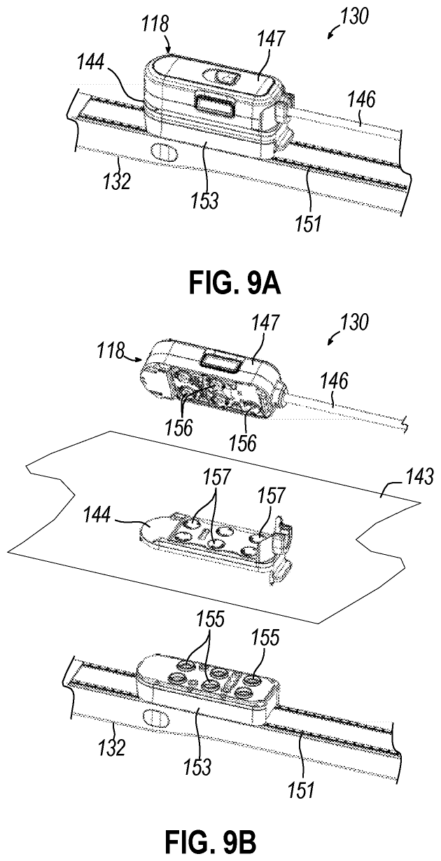

FIGS. 9A-9B depicts an example of a distal assembly 130 of a robotic manipulator, in accordance with some embodiments. FIG. 9A is an enlarged view of distal assembly 130 with instrument 118 coupled to tool driver 132. FIG. 9B depicts the distal assembly 130 in decoupled configuration, with instrument 118 detached from tool driver 132 to illustrate various interfaces therebetween. Distal assembly 130 may be used in connection with any of the robotic manipulators described herein, including, for instance, robotic manipulators 115R, 115F as seen in FIGS. 7-8. Instrument 118 may be configured in accordance with any of the instruments described herein, including, for instance, flexible and rigid instruments 118R, 118F.

As illustrated, distal assembly 130 can include a tool driver 132 coupled with instrument 118 mounted thereon. The tool driver 132 may include an elongate track 151 (also referred to herein as a “stage”) having longitudinal guides, and an instrument carriage 153, which is slidingly engaged with elongate track 151 and the longitudinal guides. The instrument carriage 153 provides an instrument holding portion configured to receive an instrument base 147 of instrument 118. An instrument base is sometimes referred to herein as a “handle” because it may be a part of the instrument configured to be held by the robot and/or a human user. Instrument carriage 153 can move along the elongate track 151 to thereby advance or retract the instrument 118.

Instrument base 147 may be coupled to the instrument carriage 153 through an adapter 144. Adapter 144 can be coupled to a drape 143, such that the adapter 144 and drape 143 provide a barrier that separates the robotic manipulator (which may be capital equipment) from the instruments (which may be consumable equipment). Such a barrier may help maintain cleanliness for the robot or sterility within the field where instruments interact with the patient. The barrier or drape 143 may include portions that extend over the robotic manipulator, robotic arm, and/or portions of the surgical robot. In some variations, instrument base 147 can be coupled to the instrument carriage 153 or tool driver 132 directly or without an adapter or barrier.

The tool driver 132 may actuate movements or functions of the instrument 118 mounted thereon. For example, tool driver 132 can actuate mechanisms in the housing of the instrument base 147 to actuate the instrument tip, such as through a cable system (e.g., pull wires) manipulated and controlled by actuated drives. The tool driver 132 may include different configurations of actuated drives. For example, as seen in FIG. 9B, instrument carriage 153 can include a set of drive outputs 155, which may engage a set of complementary drive inputs 156 on the instrument base 147 of instrument 118. The drive outputs 155 may be configured to engage the drive inputs 156 on the instrument 118 through intervening drive couplers 157 on the adapter 144, allowing the adapter to maintain a barrier while transferring torque or actuation forces from the tool driver 132 to the instrument 118. The drive outputs 155 may include, for example, rotary discs, each coupled to a corresponding actuator that can include a motor (and optionally a gear transmission and/or encoder). The drive outputs 155, drive inputs 156, and drive couplers 157 may each include various engagement features to facilitate engagement and mating between the corresponding inputs, outputs, and couplers to facilitate torque or force transfer for actuation. For example, each of the drive outputs 155, drive inputs 156, and/or drive couplers 157 may include a set of teeth, dogs, or notches that complement each other and mate with each other so that, when engaged and actuated by the tool driver 132, the engaged set of inputs, outputs, and couplers move in unison. The drive outputs 155 may be arranged in any suitable manner. For example, as seen in FIG. 9B, the tool driver 132 may include six rotary drives arranged in two rows, extending longitudinally along the instrument carriage 153. In some variations, the instrument carriage 153 includes more or fewer drive outputs, drive outputs positioned in different arrangements, and/or linear drive outputs instead of rotary discs.

Different procedures, port arrangements, or anatomical sites can influence different poses or positions of robotic manipulators to provide the desired access to the patient. For instance, different laparoscopic procedures may involve different arrangements of ports across different locations or quadrants of a patient's abdomen, influencing different robotic arm positions or poses that provide desired reach to patient's internal anatomy through such ports. An upper GI procedure can, for instance, involve introducing one or more instruments through a patient's mouth, while a lower GI procedure can involve introducing instrument(s) through a patient's perineum, influencing positioning of robotic arms near the head or the foot of a table, respectively. In some instances, hybrid procedures can involve different permutations or combinations of such access points, including both natural orifice access points and incision-based access points concurrently.

It is challenging to deploy arms in certain configurations with existing architectures. For instance, when the base of the robotic arm is mounted to a fixed side of an operating table, it is challenging to deploy laparoscopic arms into 3 +1 configurations (with three arms on one side of the table) as there is no easy way to transition arms from one side of a table to the other without reaching under or over and using up lots of the arm's reach. It is also challenging to add an arm for endoluminal access that can reach both the mouth and perineum, as it is challenging for such an arm sweep through space occupied by other laparoscopic arms having fixed base positions.

Arm deployment systems and methods are described below that facilitate various adaptable configurations or positions of robotic arms with respect to a patient or operating table, allowing the robotic arms to be readily repositioned in different ways across different procedures to access patient anatomy from optimal arm positions. As seen in the following examples, robotic arms can be moved onto a large ring that can be positioned around a column. The arms can be mounted on bearing carriages that can traverse around the ring independently. In some embodiments, four robotic arms (e.g., four laparoscopic arms) are mounted on the ring. In some embodiments, a fifth robotic arm (e.g., an endoscopic arm) is mounted on the ring and can also traverse around the ring independently.

Such configurations can allow all arms to be independently moved around the ring. The ring may be mounted distal of table top lift, tilt, and Trendelenburg degrees of freedom of a surgical table, allowing the arms to move with the table and need less reach compared to other approaches. In some variations, the ring does not have any Z-height control independent of the table top, which can improve performance compared to other approaches. The deployment system may also allow the arms on the system to be dynamically reconfigured to allow for an arbitrary number of lap arms on each side of the table (e.g., both 2+2 and 3+1 setups are possible) while also allowing an endo arm to sweep from mouth to perineum. This can enable some new combined endoscopic and laparoscopic robot setups while also expanding system possibilities.

It is challenging to make electrical connections to robotic arms while allowing a high range of motion and a large through bore for integration with a table as described above. Technologies are also described below that facilitate transfer of electrical power and/or data to robotic arms in a robust and reliable fashion as the arms move along the ring. Each arm base carriage can be outfitted with a spring-loaded spool of cable that can pay out as the arm moves and wrap around a cylindrical center of the column. Each spool of cable can have a small capsule slip ring in the middle to make the electrical connection from the spinning spool to the arm base carriage. The spools can be spring-loaded with constant torque or power springs to allow them to maintain tension. This cabling system works well with a large through bore in a surgical table column and also allows for multiple arm carriages to move independently while sharing some of the same volume for electrical cabling, as the cables can stack on top of each other in the same radial cross section.

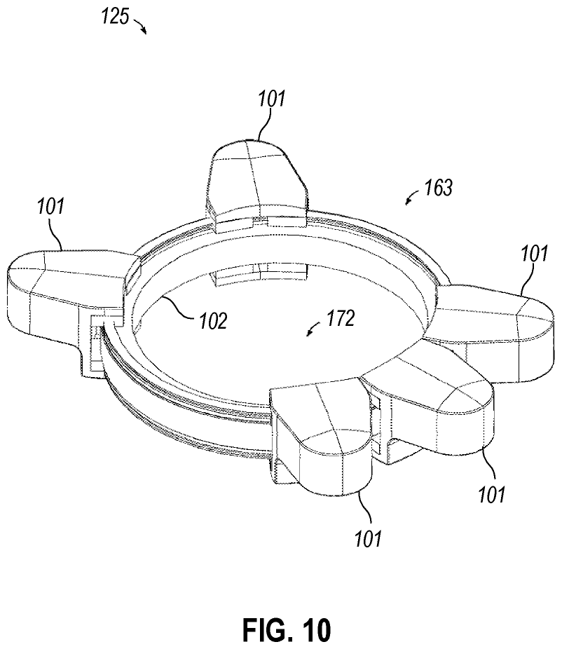

FIG. 10 depicts a perspective view of an example of a deployment system 125, in accordance with some embodiments. As illustrated, deployment system 125 includes arm support 163 and multiple arm carriages 101 configured to move along the arm support 163. Each arm carriage 101 may be coupled to a respective robotic manipulator 115 (115R, 115F) (FIGS. 6-8) such that movement of the arm carriage 101 along the arm support 163 moves a position of the respective robotic manipulator relative to patient 114 or table top 160 (FIG. 6). For instance, an arm base 139 (FIG. 7-8) of a robotic arm may be mounted to each arm carriage 101 so that the arm carriage 101 can dynamically reposition the arm base 139 as the arm carriage 101 moves along the arm support 163.

In the illustrated example, deployment system 125 includes five arm carriages 101 mounted to the arm support 163 to support five corresponding robotic manipulators (e.g., four laparoscopic manipulators 115R and one endoscopic manipulator 115F as seen in FIG. 6). In some variations, deployment system 125 includes four arm carriages 101 mounted to the arm support 163 to support four corresponding robotic manipulators (e.g., four laparoscopic manipulators 115R without any additional endoscopic manipulator 115F). In some variations, deployment system 125 includes more or fewer arm carriages to support more or fewer robotic arms or manipulators. For instance, deployment system 125 can include one, two, three, four, five, six, or more arm carriages 101 as appropriate for a given application.

Arm support 163 includes a support body 102, which can provide a main body that structurally supports arm carriages 101 (and robotic arms mounted thereon). Support body 102 can also provide a housing that houses components of the deployment system. In the illustrated example, support body 102 and arm support 163 are configured as a ring, which may be mounted to surgical table 116 and positioned about portions of column 162 (FIG. 6). This ring-shaped configuration can facilitate extension of electronic or mechanical components of the surgical table 116 through the center opening 172 of the ring, while allowing each arm carriage 101 to be independently positioned about the surgical table 116 by moving along a circular path of the ring to any circumferential position of the ring (e.g., movement to any position along 360 degrees of the ring). In some variations, deployment system 125 can employ different shapes for the arm support and/or more constrained movement, such that arm carriage(s) 101 can move along a non-circular path and/or move less than a full 360 degrees relative to the surgical table 116. In some variations, a single arm carriage can support two or more robotic arms, such that the two or more arms move in unison with movement of the arm carriage, and such that the two or more arms move together along the arm support instead of independently relative to each other.

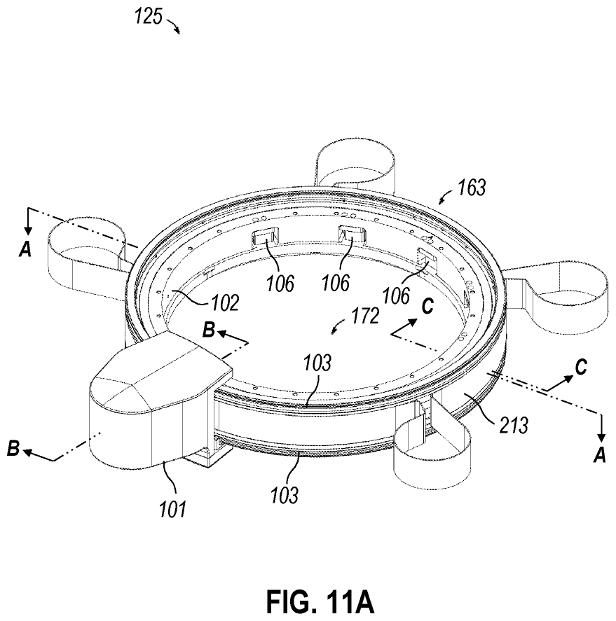

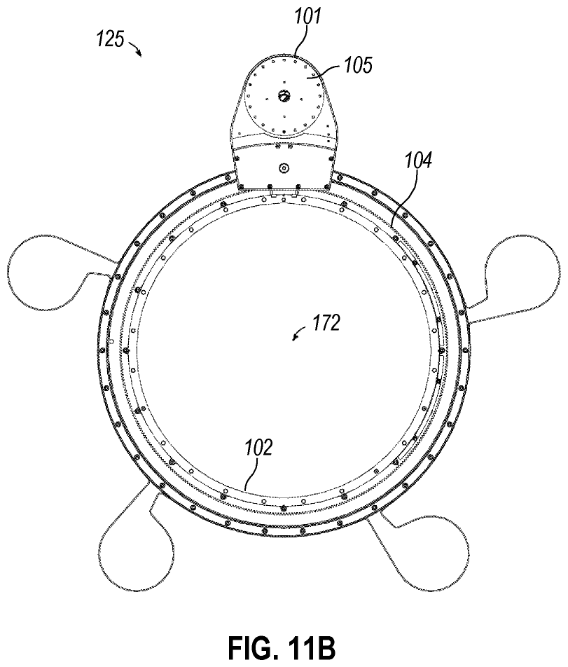

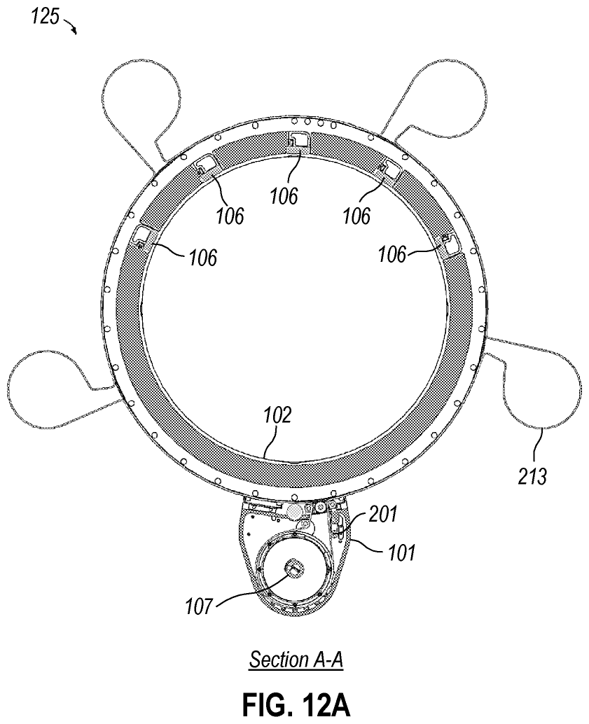

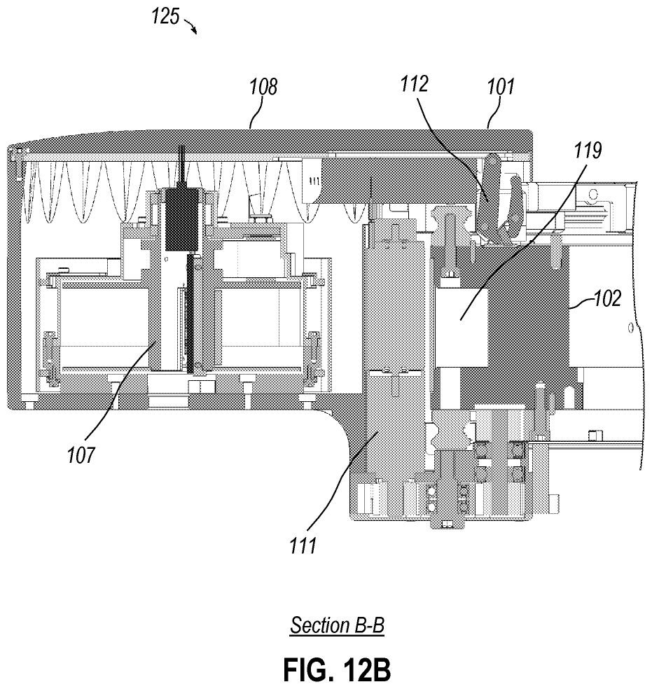

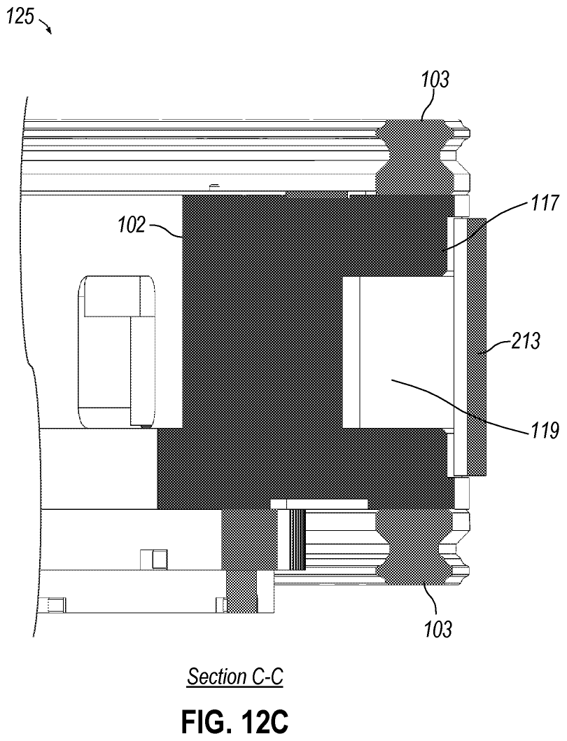

FIGS. 11A-12C depict various views of an assembly of deployment system 125. FIGS. 11A-11B depict deployment system 125 with portions hidden to illustrate internal or functional components of the system, where FIG. 11A is a perspective view and FIG. 11B is a bottom view. FIGS. 12A-12C depict section views of deployment system 125 as seen in FIG. 11A, where FIG. 12A a top view corresponding to cross section A-A, FIG. 12B is a right side view corresponding to cross section B-B, and FIG. 12C is a front view corresponding to cross section C-C.

Arm support 163 includes a track 103 that is fixed to the support body 102 and provides a path along which each arm carriage 101 can travel. As illustrated, track 103 includes a set of parallel rails extending along the path of travel for the arm carriages 101. Each arm carriage 101 can be movably mounted to track 103 (e.g., via bearing carriages) so that the arm carriage 101 can travel along the track 103. Track 103 can include spaced apart rails to distribute loads from the weight of arm carriage 101 and each robotic arm mounted thereon. In FIGS. 11A-12C, a single arm carriage 101 is shown, with other arm carriages removed. Each arm carriage 101 can be configured similarly to the arm carriage 101 as seen in FIGS. 11A-12C.

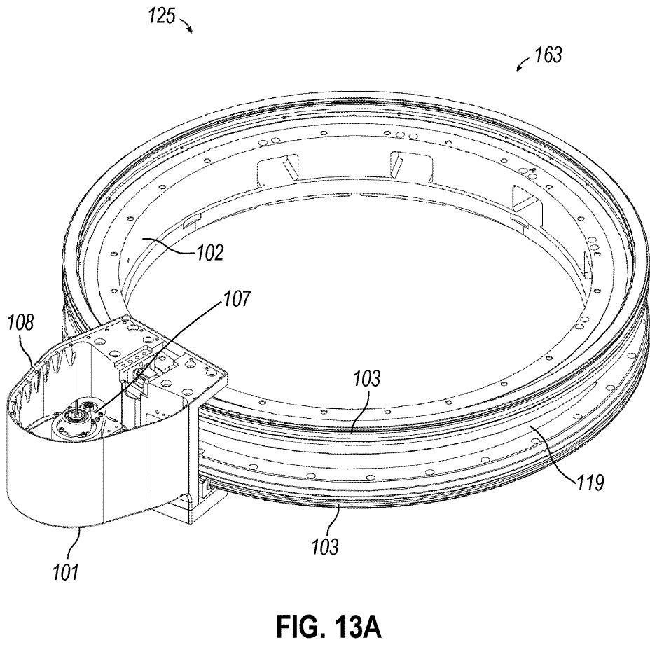

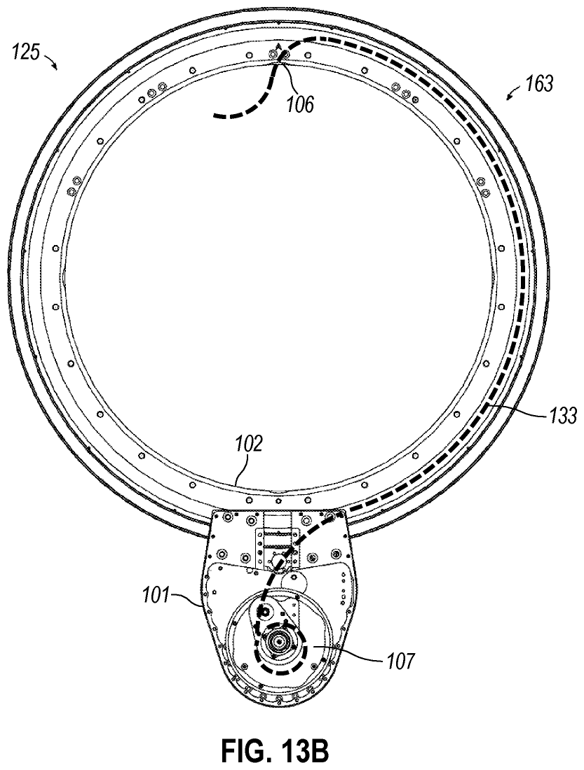

As seen for example in FIG. 12B, arm carriage 101 includes a cable winder 107, a carriage actuator 111, and a brake 112. Cable winder 107 is configured to spool or unspool (e.g., pay out or wind up) an electrical cable coupled to the arm carriage 101 as the arm carriage 101 moves along the arm support 163. Carriage actuator 111 is configured to drive movement of the arm carriage 101 along the arm support 163. Brake 112 is configured to engage the arm support 163 to brake the arm carriage 101 relative to the arm support 163 (e.g., inhibit movement of the arm carriage 101 or hold the arm carriage stationary). These assemblies can be carried or retained at least partially within carriage housing 108. These assemblies and sub-systems are further described below with respect to FIGS. 13A-19D.

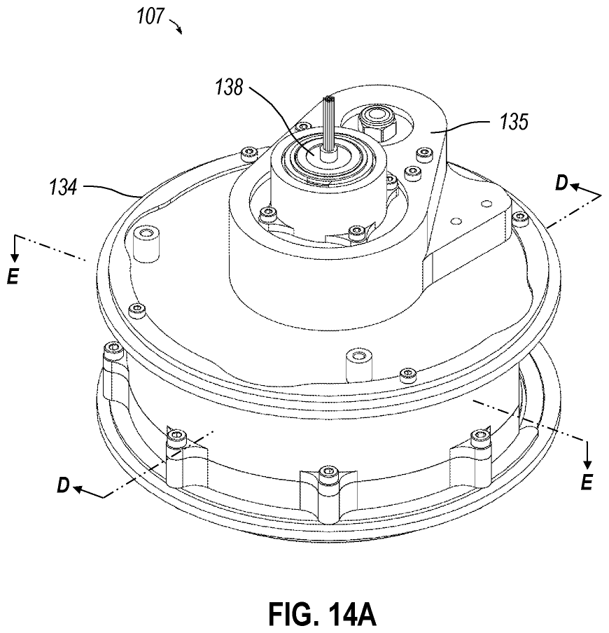

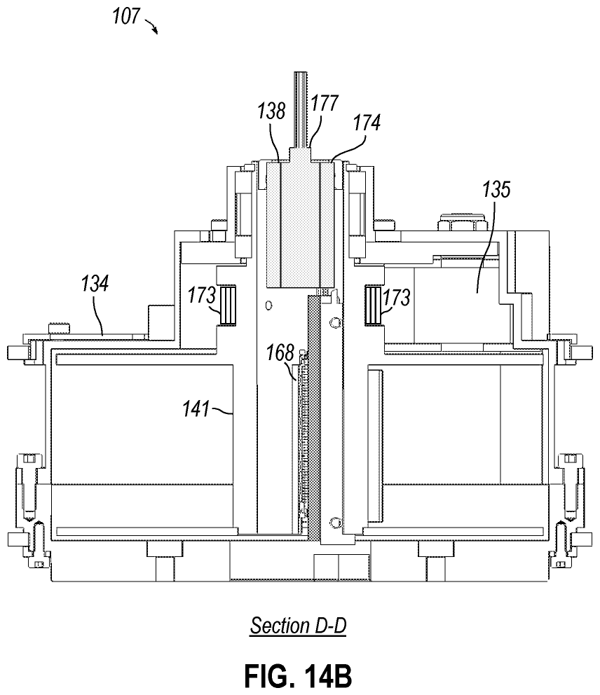

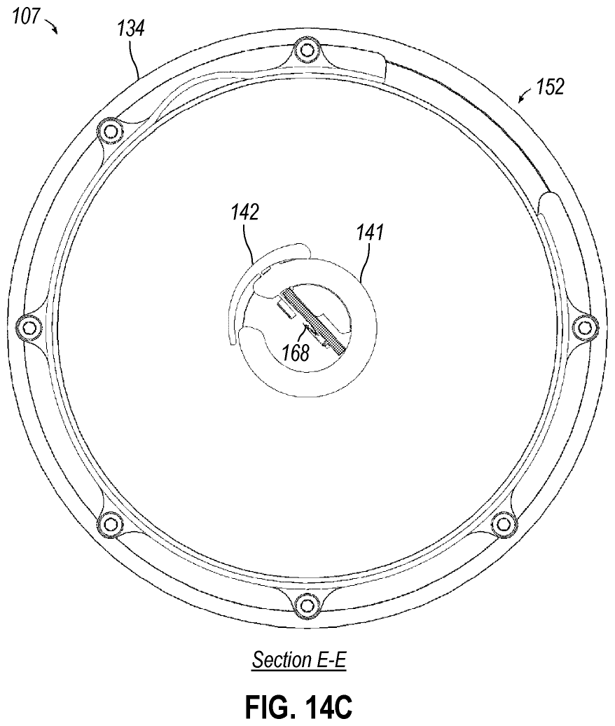

FIGS. 13A-15D depict various views of an assembly and operation of cable winder 107, which operates to spool and unspool an electrical cable as the arm carriage moves along the arm support 163. FIGS. 13A-13B depict views of deployment system 125 with some covers and internal components hidden, where FIG. 13A is a perspective view and FIG. 13B is a top view. FIGS. 14A-14C depict various views of cable winder 107 as seen in FIG. 13A-13B, where FIG. 14A is a perspective view, FIG. 14B is a right side view corresponding to cross section D-D of FIG. 14A, and FIG. 14C is a top view corresponding to cross section E-E of FIG. 14A.

Cable winder 107 (also referred to herein as a “cable winder assembly”) is mounted to arm carriage 101 and retained in carriage housing 108 such that the cable winder 107 moves with the arm carriage 101 along track 103. Cable winder 107 includes a spool 141 rotatably coupled to arm carriage 101 and attached to a cable 133 such that spool 141 pays out or winds up cable 133 as the arm carriage 101 moves along the track 103.

A cable path 119 extends along the path of travel of the arm carriage 101 and alongside track 103. As illustrated, cable path 119 sits in a recess in the support body 102 which can extend circumferentially around the support body 102. A first portion or end of the cable 133 can be attached to the moving spool 141 while a second portion of end of cable 133 can be attached to a cable attachment 106 (e.g., a cable clamp or connector) at a fixed point along or relative to cable path 133. FIGS. 13A-13B depict a single cable 133, but as further described herein, cable path 119 or support body 102 can include multiple cable attachments 106 that respectively attach to multiple cables 133 corresponding to multiple arm carriages 101.

Cable winder 107 includes a winder housing 134, which can be fixedly coupled or mounted to the carriage housing 108. Spool 141 can be rotatably mounted or coupled to the winder housing 107 such that spool 141 can rotate (spin about its axis) relative to winder housing 107 and arm carriage housing 108 as the arm carriage 101 moves along the arm support. In some variations, spool 141 is spring-loaded to urge rotation of the spool and retraction of the cable 133 in concert with movement of the arm carriage 101.

As illustrated, cable winder 107 can include a spring housing 135, which retains a winder spring 173 of the cable winder 107. Here, spring housing 135 is retained in an upper portion of winder housing 134. Winder spring 173 is operatively coupled to the spool 141 and applies torque to spool 141 to maintain tension on cable 133 and to wind up spool 141 as the arm carriage 101 moves in a direction toward cable attachment 106. Winder spring 173 can include, for instance, a constant torque spring or power spring, which can apply a constant or continuous force to spool 141 relative to winder housing 134. Winder spring 173 can be fixed or coupled at one end to the spinning spool 141 and fixed or coupled at another end to the spring housing 135 such that the force applied to spool 141 by winder spring 173 drives rotation of spool 141.

As spool 141 spins, cable 133 winds up onto or pays out from spool 141 depending on the direction of travel of the arm carriage 101. As the arm carriage 101 is moved towards cable attachment 106 in the direction of tension on cable 133 (e.g., counterclockwise), winder spring 173 urges rotation of the spool 141 that causes spool 141 to wind up and take in cable 133 from cable path 119 into the cable winder 107 through cable opening 152 that is formed in the winder housing 134, in concert with the movement of the arm carriage 101. As the arm carriage 101 moves in an opposite direction, away from the cable attachment 106 and away from the direction of tension on cable 133 (e.g., clockwise), a driving force applied to arm carriage 101 can overcome the spring force of winder spring 173 to unwind or pay out the cable 133 from cable winder 107 onto cable path 119 through cable opening 152, in concert with the movement of the arm carriage 101.

Cable 133 can be an electrical cable (e.g., a ribbon cable) that carries power and/or data to arm carriage 101 and/or a robotic arm mounted to the arm carriage 101. A first end or portion of cable 133 can be attached to spool 141 and a second end or portion of cable 133 can be attached to cable path 119. The second portion of cable 133 can, for instance, be further electrically coupled to system components to send or receive power or data for controlling the robotic arm (e.g., signals from control system 145 via electrical signal lines that can extend through portions of column 162 or base 161 as seen in FIGS. 1 and 6). Cable 133 can, for instance, be clamped to cable attachment 106 and continue to extend through a center cylinder or column, or cable 133 can terminate at an electrical connector or socket at cable attachment 106, such that further electrical cables or circuit boards electrically connected to the cable attachment electrical send or receive signals through a center cylinder or column.

As illustrated, cable winder 107 includes a slip ring 138 (e.g., a capsule type slip ring) that can electrically couple the cable 133 to a robotic arm or manipulator mounted to the arm carriage 101 and maintain electrical connection therebetween as the spool 141 rotates. For instance, as illustrated, cable 133 can be attached to the spinning spool 141. Cable 133 can be electrically connected to spool 141 via electrical connector 168 and mechanically fixed to spool 141 via cable clamp 142. Slip ring 138 can have a first part 174 (e.g., stator) electrically and mechanically connected to electrical connector 168, and a second part 177 (e.g., rotor) electrically and mechanically connect to the robotic arm. First part 174 and second part 177 are rotatable relative to each while maintaining electrical connection therebetween, thereby permitting the spool 141 to rotate relative to the robotic arm as the arm carriage 101 moves, without losing electrical connection to the robotic arm or unduly tangling and twisting the cable.

Cable 133 can be seated in a recess in the support body 102 formed by cable path 119. cover band 213 can cover the cable 133 and/or cable path 119 to provide mechanical protection to cable 133 and/or cable path 119, and/or provide an aesthetically pleasing enclosure for the deployment system 125. Cover band 213 can be seated over the cable path 119 and include a conveyor belt material to allow cover band 213 to slide through the arm carriage 101 as the arm carriage 101 moves along the track 103. As seen for instance in FIGS. 11A and 12A, cover band 213 can be routed through arm carriage 101 outside of the cable winder 107 or spool 141 to maintain coverage over the cable 133 without interfering with the operation of the spool. Cover band 213 can be seated in a band recess 117 and may extend around the entire cable path 119 and arm support 163. Arm carriage 101 may include a tensioner 201 to keep the cover band 213 taut as the arm carriage 101 slides through the cover band 213. Arm carriage 101 can include one or more pulleys and/or bushing surfaces to facilitate movement of the cover band 213 through the arm carriage 101.

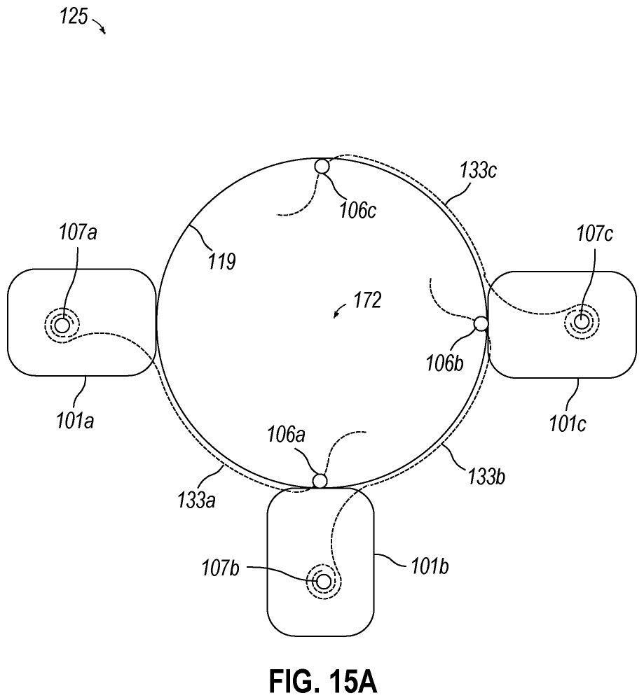

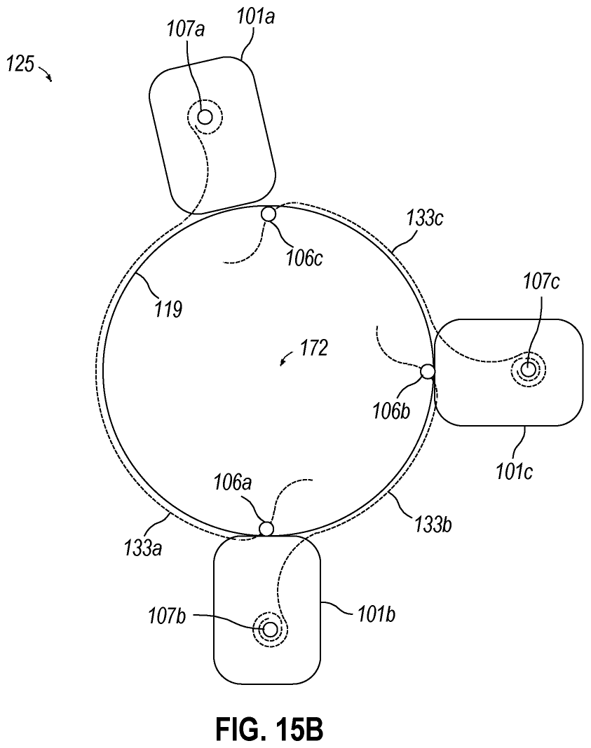

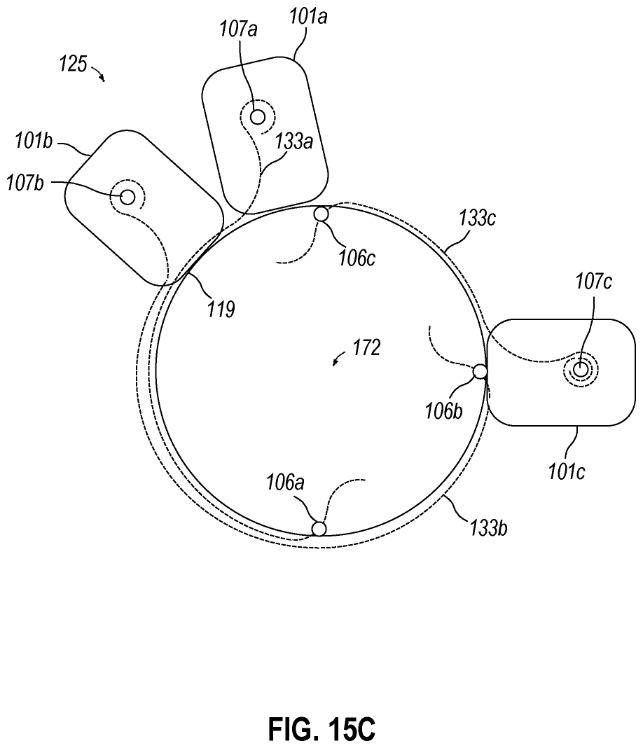

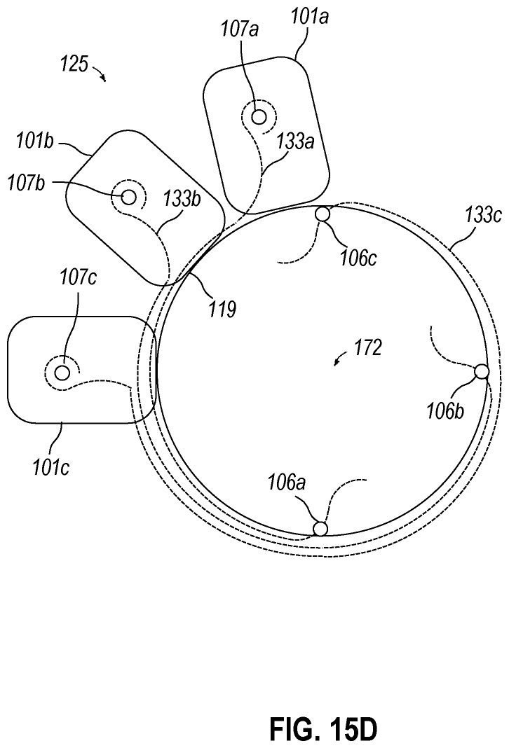

FIGS. 15A-15D depict schematic views of multiple cable winders 107a-c in operation in various states, where FIG. 15A depicts a first state of operation, FIG. 15B depicts a second state of operation, FIG. 15C depicts a third state of operation, and FIG. 15D depicts a fourth state of operation.

In the illustrated example, three cable winders 107a-c respectively corresponding to three arm carriages 101a-c and three cables 133a-c are shown. The various states in FIGS. 15A-15D depict an example of how deployment system 125 can stack electrical cables 133a-c on top of each other on the cable path 119 as the arm (e.g., about central opening 172). Here, cable path 119 is circular in accordance with a ring-shaped arm support as described above. Accordingly, the cable path 119 can correspond to a center cylinder positioned about central opening 172.

FIG. 15A depicts deployment system 125 in a first state where first arm carriage 101a, second arm carriage 101b, and third arm carriage 101c are each in initial positions. First arm carriage 101a includes a first cable winder 107a coupled to a first cable 133a that extends along cable path 119 and is attached to a first cable attachment 106a, which is located at a first fixed point along the cable path 119. Second arm carriage 101b includes a second cable winder 107b coupled to a second cable 133b that extends along cable path 119 and is attached to a second cable attachment 106b, which is located at a second fixed point along the cable path 119. Third arm carriage 101c includes a third cable winder 107c coupled to a third cable 133c that extends along cable path 119 and is attached to a third cable attachment 106c, which is located at a third fixed point along the cable path 119. Each of these components can be similar to the arm carriage 101, cable winder 107, cable 133, cable attachment 106, and cable path 119 as described above.

FIG. 15B depicts deployment system 125 in a second state where first arm carriage 101a is moved to an updated position along cable path 119, while second arm carriage 101b and third arm carriage 101c remain in their initial positions associated with the first state. Upon movement of the first arm carriage 101a to its updated position, first cable winder 107a unspools first cable 133a such that the first cable 133a pays out onto the cable path 119. Here, first cable 133a wraps further around the center cylinder and about central opening 172 compared to its initial position.

FIG. 15C depicts deployment system 125 in a third state where first arm carriage 101a remains in its updated position, second arm carriage 101b is moved to an updated position along cable path 119, and third arm carriage 101c continues to remain in its initial position. Upon movement of the second arm carriage 101b to its new position, second cable winder 107b unspools second cable 133b such that the second cable 133b pays out onto the cable path 119. Here, second cable 133b wraps or is let out further around the center cylinder and about central opening 172. Second cable 133b also wraps or is let out onto the first cable 133a, such that the second cable 133b is stacked onto the first cable 133a.

FIG. 15D depicts deployment system 125 in a fourth state where first arm carriage 101a and second arm carriage 101b remain in their updated positions, and third arm carriage 101c is also moved to an updated position. Upon movement of the third arm carriage 101c to its new position, third cable winder 107c unspools third cable 133c such that the third cable 133c pays out onto the cable path 119. Here, third cable 133c wraps or is let out further around the center cylinder and about central opening 172. Third cable 133c also wraps or is let out onto the second cable 133b, which is in turn stacked onto first cable 133a, such that the third cable 133c, second cable 133b, and first cable 133a are stacked in a series.

As described above, the deployment system 125 can allow multiple arm carriages 101a-c to move independently while maintaining robust and reliable electrical connections to the carriages (and robotic arms mounted thereon). The carriages may share some of the same volume for electrical cabling for a compact configuration without tangling, for instance, by stacking the multiple cables 133a-c on top of each other in the same cross section or radial cross section. By arranging the arm support 163 in a ring-shaped or circumferential configuration about a surgical table or column 162 of a surgical table (FIG. 6) the deployment system 125 can allow each arm carriage to be positioned with a high range of motion to various dynamic configurations relative to the table top 160 or patient 114. For instance, in some configurations, each arm carriage 101 may be positioned independently along the common track in any location up to a full 360 degrees of the arm support 163.

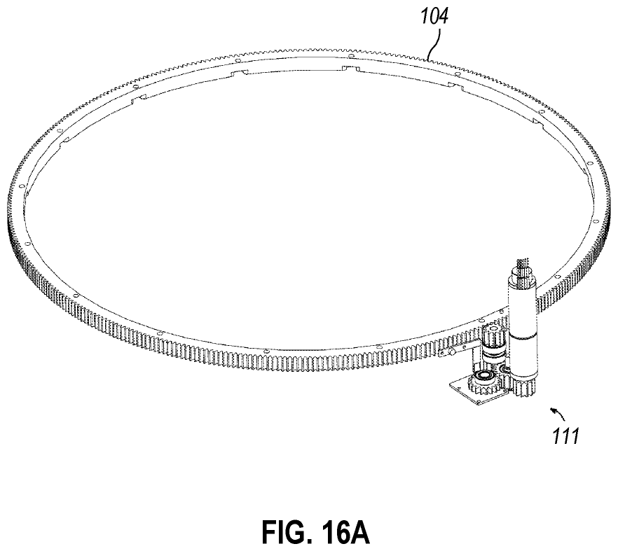

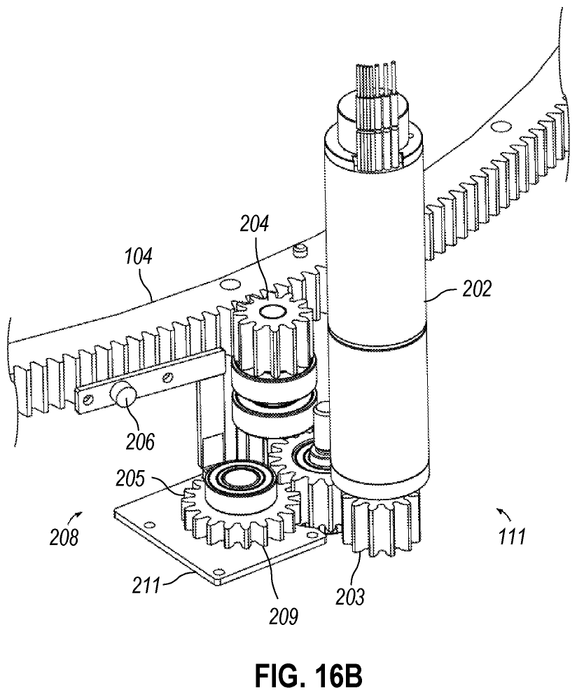

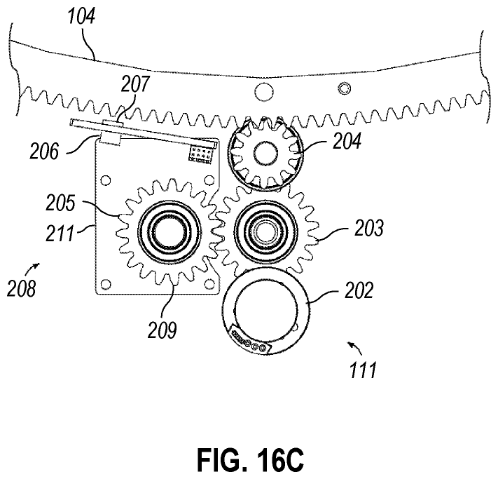

FIGS. 16A-16C depict an example of a carriage actuator 111 and actuation system that can drive movement of the arm carriage 101 along the track 103. FIG. 16A is a perspective view of components of the carriage actuator 111 and actuation system isolated with other portions of deployment system 125 hidden. FIG. 16B is an enlarged view of the perspective view seen in FIG. 16A, and FIG. 16C is a top view of the enlarged view seen in FIG. 16C. Portions of the carriage actuation system are also seen, for instance, in FIGS. 11B and 12B.

Carriage actuator 111 (also referred to herein as a “carriage actuator assembly”) is retained in the arm carriage 101 such that carriage actuator 111 moves with the arm carriage 101 as the carriage actuator 111 drives movement of the arm carriage 101 along the track 103. Carriage actuator 111 can include a motor 202 operatively coupled to ring gear 104 via a pinion gear 204 that is meshed with the ring gear 104. Ring gear 104 can be fixedly coupled to the support body 102 alongside the track 103 so that as motor 202 drives rotation of the pinion gear 204, the arm carriage 101 traverse the track 103 along the same path of travel. Motor 202 can drive the pinion gear 204 in a first direction to drive movement of the arm carriage 101 in a first direction (e.g., clockwise), and motor 202 can drive the pinion gear 204 in a second, opposite direction to drive movement of the arm carriage 101 in a second, opposite direction (e.g., counterclockwise).

In the illustrated example, carriage motor 202 is operatively coupled to the pinion gear 204 via an intermediate gear train 203, which has one or more intermediate gears. In some variations, carriage motor 202 is directly coupled to the pinion gear 204 or carriage actuator 111 has different gearing arrangements. Carriage motor 202 can be fixedly coupled or mounted to carriage housing 108 and can be powered via cable 133, as described above.

As seen in FIGS. 16B-16C, carriage actuator 111 includes an encoder system 208, which can be used to determine the position of the arm carriage 101. For instance, control system 145 (FIG. 1) can use encoder system 208 to determine a position of each arm carriage 101 along the arm support 163 relative to the reference frame of the surgical robot 110 (FIGS. 1, 6) and therefore determine a position or pose of each corresponding robotic arm for kinematic calculations. Such kinematics may be useful, for instance, for collision avoidance algorithms or kinematic optimizations, among other purposes. Designing an encoder system for arm carriages 101 can be challenging, as small errors in sensing at base of the arm can lead to large errors propagating through each encoder measurement along the kinematic chain to the end effector at the distal end of the arm. In some variations, arm carriages 101 are configured to complete more than one full revolution about the ring, and it may be beneficial for the control system to know which revolution the arm carriage is currently on, in addition to the absolute position of the arm carriage relative to the ring.

In the illustrated example, encoder system 208 includes coarse encoder 205 and fine encoder 206 to employ a coarse to fine approach to the position determination. Coarse encoder 205 can detect at which gear tooth of ring gear 104 the carriage actuator 111 is currently positioned (and therefore, at which gear tooth the arm carriage 101 is currently positioned). Fine encoder 206 can detect the position of the carriage actuator 111 relative to the current gear tooth (and therefore the position of the arm carriage 101 relative to the current gear tooth).

Coarse encoder 205 includes an encoder gear 205 operatively coupled to the motor 202 such that the motor 202 drives rotation of the encoder gear 209. In the illustrated example, encoder gear 205 is meshed with one of the intermediate gears of gear train 203. Encoder gear 209 may be mounted adjacent an encoder board 211 having a sensor or electronics that detect revolutions of the encoder gear 209. The revolutions of encoder gear 209 or number of gear teeth incremented by the encoder gear 209 can correspond to the number of gear teeth on ring gear 104 incremented by carriage actuator 111 to therefore provide an incremental calculation of the current gear tooth position.

Fine encoder 206 includes a gear tooth sensor 207 which can face the teeth of the ring gear 104 to detect a relative position relative to a current gear tooth. Coarse encoder 205 and/or fine encoder 206 may, for instance, use magnetic, capacitive, or optical sensing technologies. For instance, gear tooth sensor 207 can magnetically detect the peaks and valleys of the teeth of ring gear 104 to detect the relative position of the current gear tooth.

It can be beneficial to inhibit movement of the arm carriage 101 when arm carriage 101 is not being actively repositioned. For instance, it can be beneficial to apply brakes to the arm carriage 101 to stabilize the arm base during surgical tasks performed by the robotic arms, or avoid unintended movement of the arm carriage 101 in the event of a power failure.

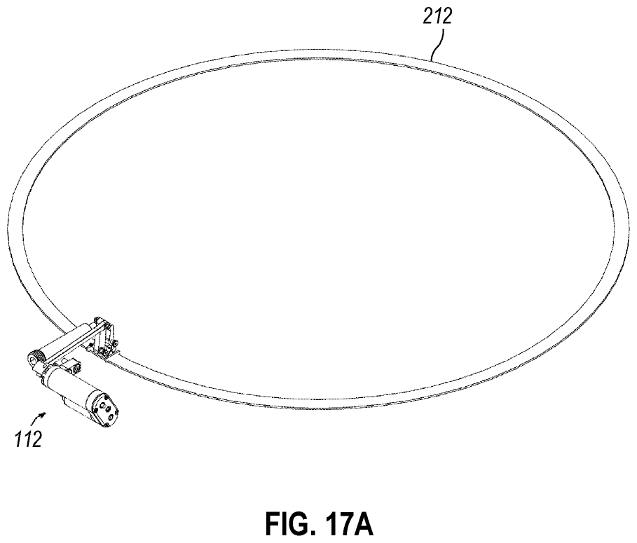

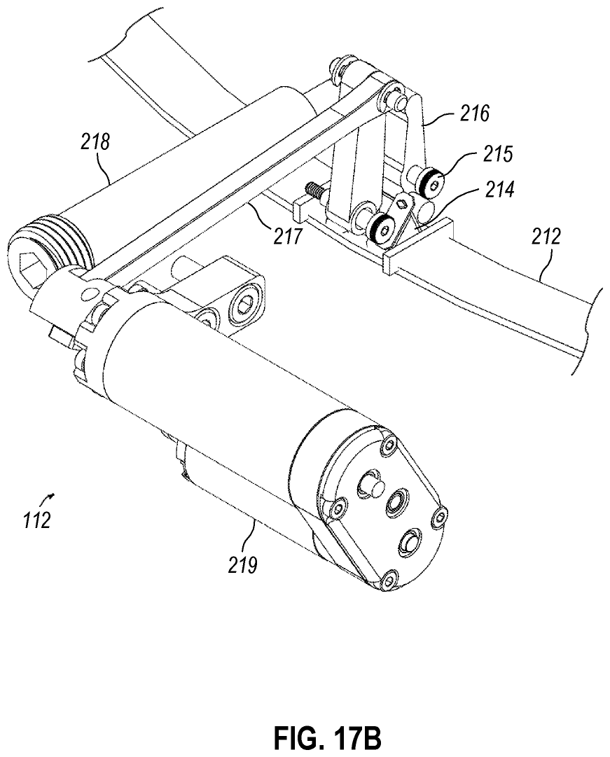

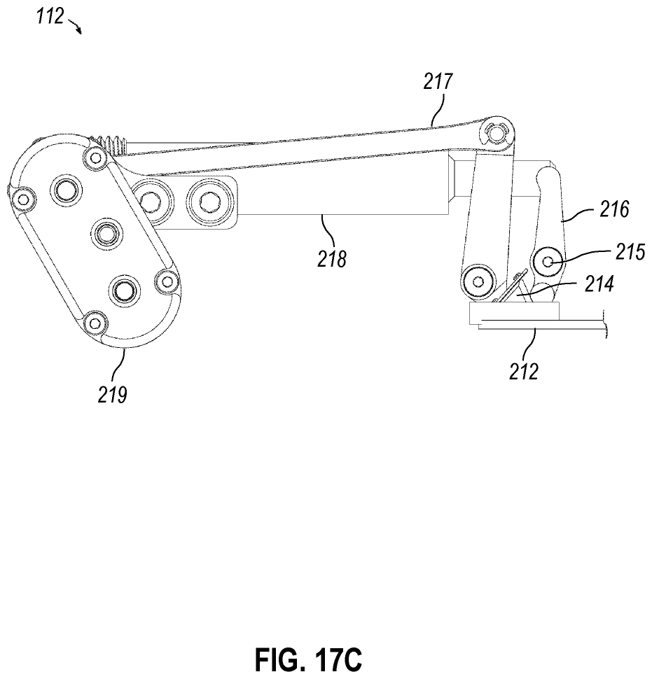

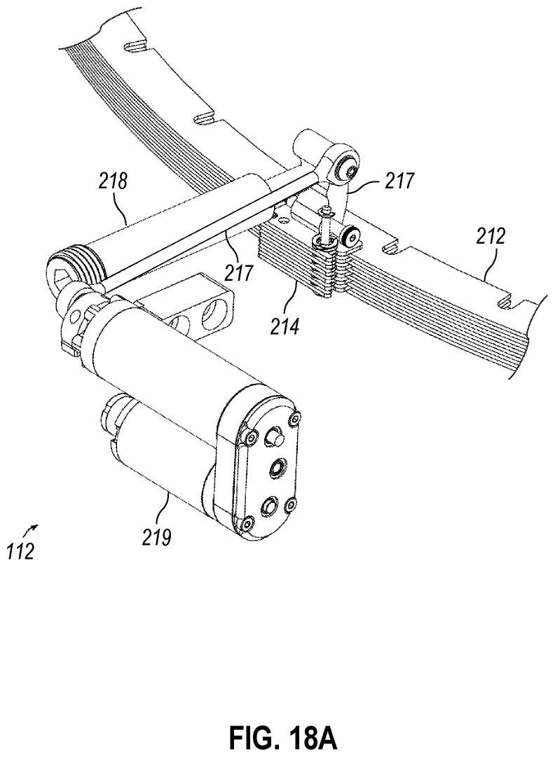

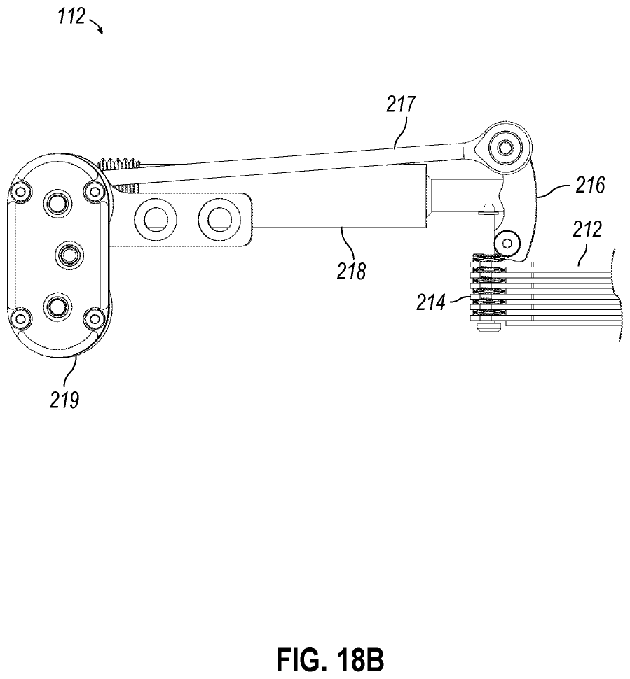

FIGS. 17A-18B depict example variations of brake 112 that can inhibit movement (e.g., to hold the arm carriage 101 stationary along the arm support). FIGS. 17A-17C depicts various views of brake 112, where FIG. 17A is a perspective view of brake 112, FIG. 17B is a enlarged view of the brake 112 as seen in FIG. 17A, and FIG. 17C is a right side view of the enlarged view seen in FIG. 17C. FIG. 18A is an enlarged perspective view of an example variation of brake 112, and FIG. 18B is a right side view of the enlarged view seen in FIG. 18C.

Brake 112 (also referred to herein as a “brake assembly”) can be retained in arm carriage 101 such that brake 112 moves with movement of the arm carriage 101. Brake 112 is configured to engage with an engagement surface 212, which can be fixedly coupled to the support body 102 alongside track 103 so that as the arm carriage 101 traverses the track, brake 112 is capable of engaging or applying brakes to the engagement surface 212 along the same path of travel. Engagement surface 212 can include, for instance, a plate or top face of support body 102. Engagement surface 212 can extend alongside the entire track 103 so that brake 112 can engage the engagement surface 212 in any location along the full range of motion of the arm carriage 101.

Brake 112 includes a movable component 214 that is urged against the engagement surface 212 by force applied by brake spring 218. When engaged with or urged against engagement surface 212, movable component 214 can inhibit movement of the arm carriage 101 relative to arm support 163 via friction force between the movable component 214 and engagement surface 212. In the illustrated example, brake spring 218 is implemented as a gas spring 218, which applies force to movable component 214 via lever 216, thereby providing mechanical advantage for enhancing the braking force applied by brake spring 218.

Brake spring 218 is biased in a direction that urges movable component 214 into engagement with the engagement surface 212. As illustrated in FIGS. 17C and 18B, brake spring 218 is biased to urge top portion of lever 216 to the right, so that lever 216 pivots about fulcrum 215 to urge movable component 214 against engagement surface 212.

Brake actuator 219 is configured to disengage brake 112 by counteracting the force applied brake spring 218. Therefore, brake 112 can be default engaged via brake spring 218 in the absence of actuation by brake actuator 219. Such a configuration can avoid unintended movement by inhibiting movement of the arm carriage 101 in the absence of power (e.g., in the event of a power failure).

Brake actuator 219 can include a motor configured to disengage moveable component 214 via actuation of linkage 217 operatively coupled to movable component 214 and/or lever 216. During actuation of arm carriage 101 to move arm carriage along the track 103 (e.g., as described above with respect to FIGS. 16A-16C), brake actuator 219 can be actuated in concert with carriage actuator 111 so that brake actuator 219 disengages brake 112 to permit movement of the arm carriage 101 while carriage actuator 111 drives movement of the arm carriage. Brake actuator 219 can then release the movable component 214 to engage brake 112 upon completion of the movement of the carriage.

FIGS. 17A-17C depict an example variation where movable component 214 is implemented as a wedge. Brake spring 218 urges the wedge between the engagement surface 212 and a complementary wedge surface of the brake assembly that moves with the arm carriage 101. As illustrated in FIG. 17C, brake spring 218 urges the wedge to the left via lever 216 to wedge the movable component 214 between the lower engagement surface 212 (which is fixed to the support body) and an upper left angled surface that.

FIGS. 18A-18B depict an example variation where movable component 214 and engagement surface 212 are implemented as a series of interleaved plates, which can enhance surface of the brake engagement to enhance a friction force of the brake 112. Brake spring 218 urges a first set of plates (of movable component 214) against a second set of plates (of engagement surface 212) via movement of the lever 216. As with the engagement surface 212 in the variation of brake 112 seen in FIG. 17A, the interleaved plates of engagement surface 212 can be fixedly coupled to the support body 102 and extent alongside the track 103. As illustrated in FIG. 18B, brake spring 218 urges the plates of movable component 214 downward via lever 216 to press the plates of movable component 214 against complementary interleaved plates of engagement surface 212.

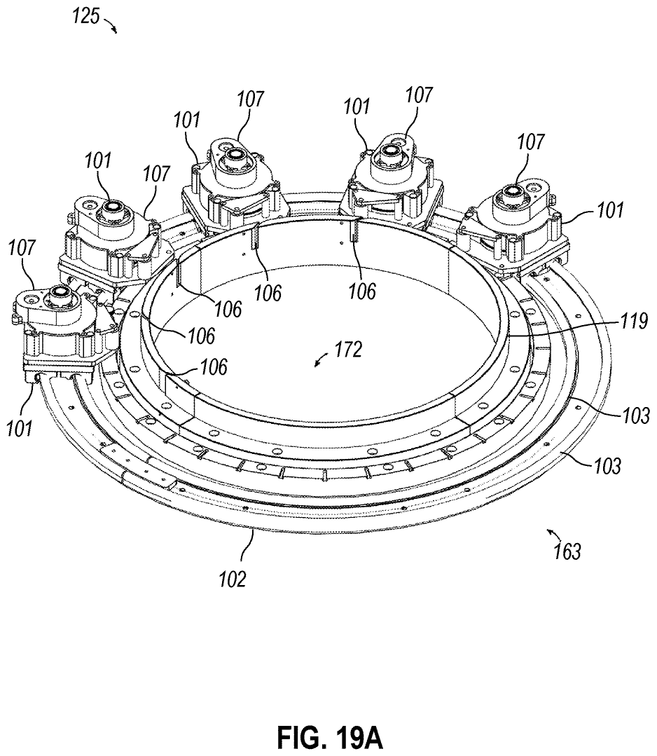

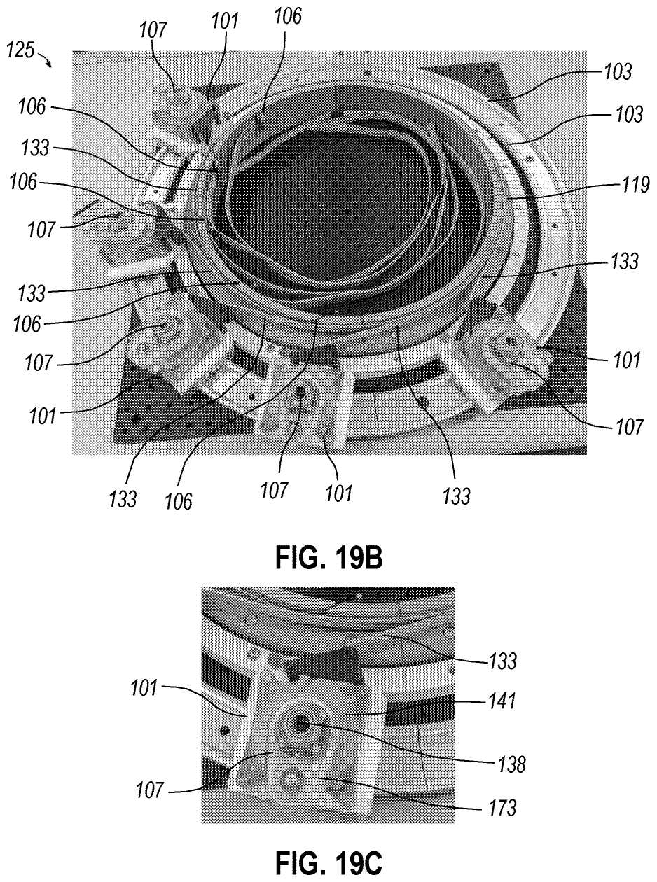

FIGS. 19A-19C depict an example variation of deployment system 125. FIG. 19A is a perspective view of deployment system 125 without cables attached. FIG. 19B is an image of deployment system 125 with cables attached. FIG. 19C is an enlarge view of an arm carriage assembly from FIG. 19B.

The example variation of deployment system 125 has a track 103 with parallel rails oriented horizontally, as opposed to vertically as seen in FIG. 10. As in the above example, cable path 119 extends alongside track 103 but is not recessed between the parallel rails as in FIG. 12C. Deployment system 125 can otherwise have similar components and operation as described above. For instance, as seen in FIG. 19B, deployment system 125 includes multiple arm carriages 101 mounted to track 103, and multiple cable winders 107 coupled to multiple corresponding cables 133 attached to fixed attachment points along cable path 119, where cables 133 stack on top of each other as the carriages move along the track. As seen in FIG. 19C, cable winder 107 or arm carriage 101 can include a spool 141 of cable 133, a slip ring 138, and a winder spring 173 that maintains tension on the spool.

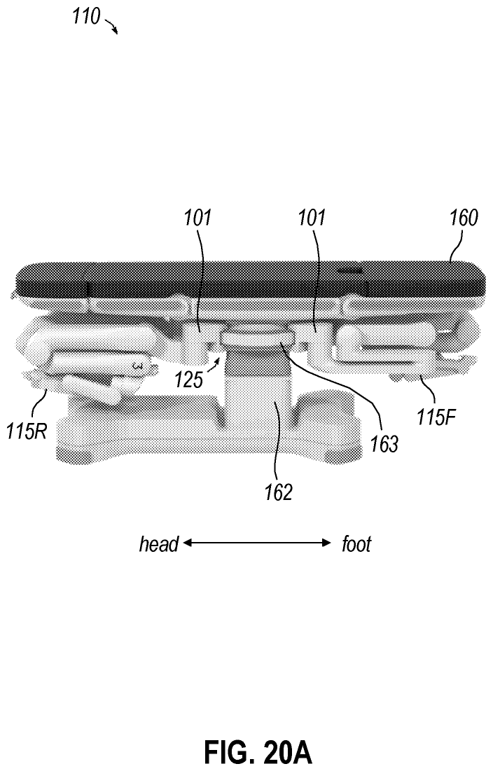

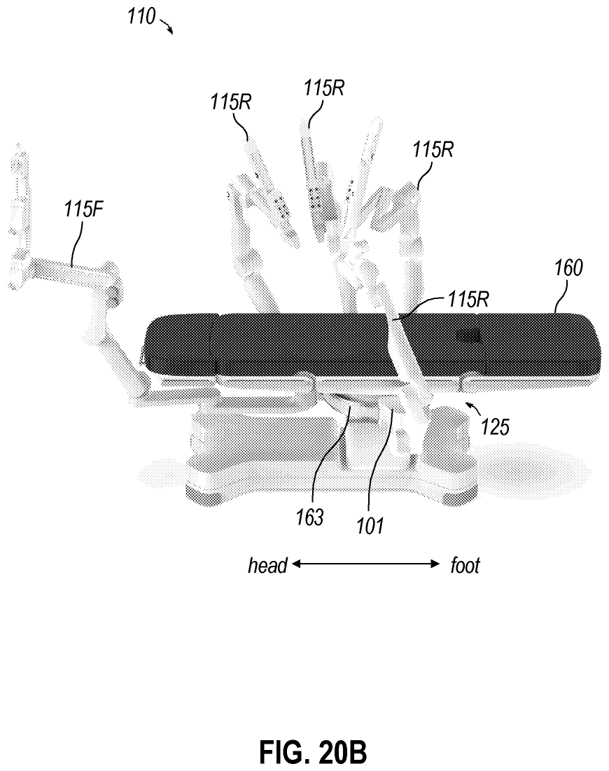

FIGS. 20A-22C depict various arm configurations using deployment system 125 in accordance with some embodiments. Deployment system 125 can be integrated with surgical robot 110, where arm support 163 is coupled to a surgical table around a portion of table column 162. Arm carriages 101 may traverse the arm support 163 and sweep around the column 162 to position robotic manipulators 115R, 115F in various configurations. As illustrated, each robotic manipulator 115R, 115F includes a robotic arm as described above with respect to FIGS. 7-8 and is referred to as a robotic arm in the description that follows. Flexible instrument robotic arm 115F is referred to as an endoscopic arm 115F, and rigid instrument robotic arm 115R is referred to as a laparoscopic arm 115R, as these arms may be respectively configured for endoscopic and laparoscopic use as described above. Arm carriages 101 are configured to support the robotic arms 115R, 115F, and arm support 163 can encircle the surgical table or column 162 to facilitate positioning the robotic arms in various configurations around the surgical table. It will be appreciated that similar or different dynamic arm configurations as those shown may be deployed for different types and combinations of robotic arms.

FIG. 20A is a perspective view of deployment system 125 with robotic arms 115R, 115F in a stowed configuration, where robotic arms 115R, 115F are folded in a compact configuration and positioned beneath table top 160. Here, the arm carriages 101 can move along the arm support 163 toward opposing positions at the head and foot of the surgical table to facilitate the stowed configuration.

FIG. 20B is a perspective view of deployment system 125 with robotic arms 115R, 115F in a deployed configuration, where robotic arms 115R, 115F are raised above the table top 160 for use. Here, laparoscopic arms 115R are configured in a 3+1 configuration, where three laparoscopic arms 115R are deployed along a first lateral side of the table top 160, and one laparoscopic arm 115R is deployed along an opposite lateral side of table top 160. Endoscopic arm 115F is deployed along a head of table top 160.

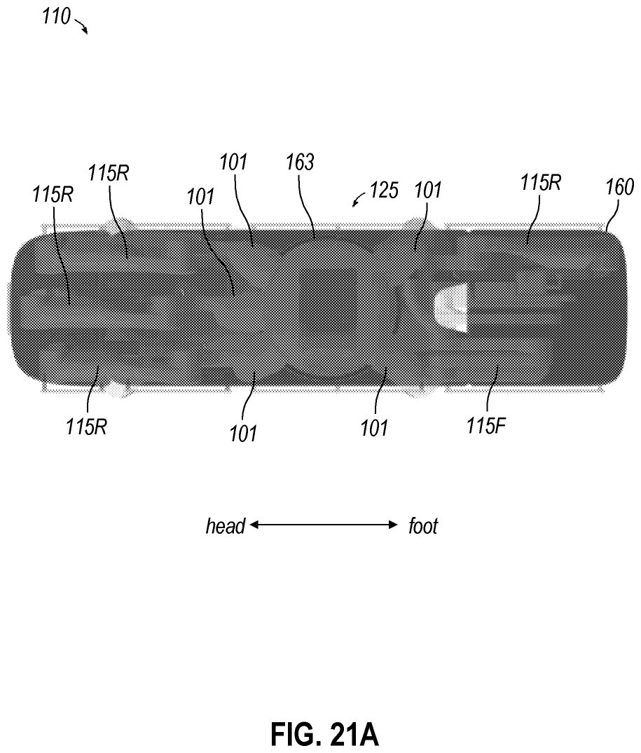

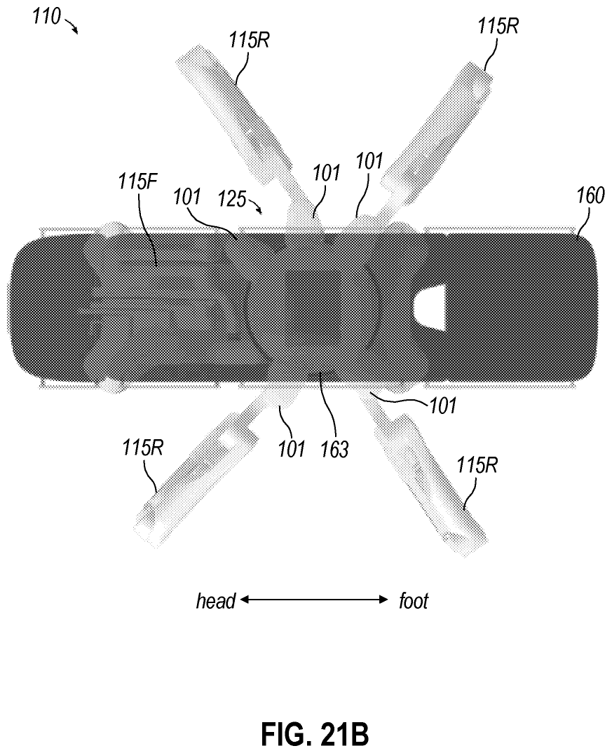

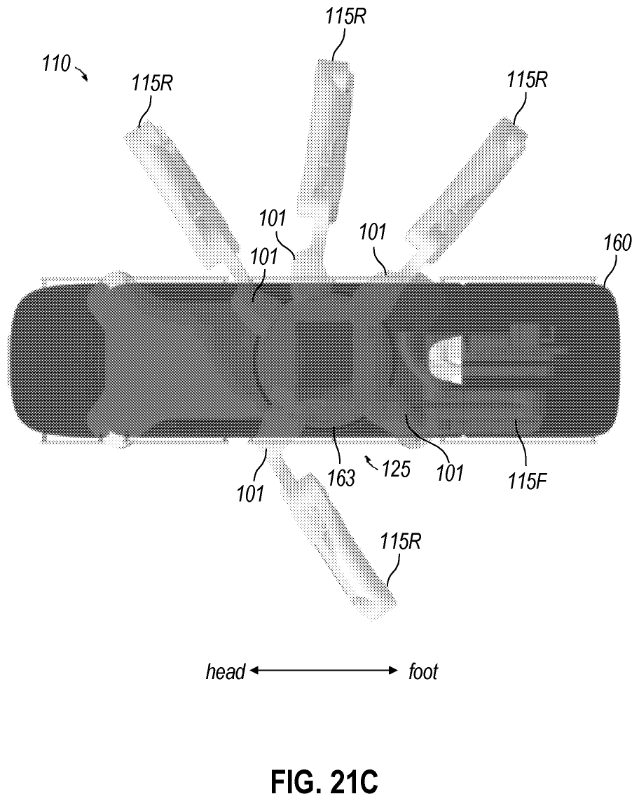

FIG. 21A is a top view of deployment system 125 with robotic arms 115R, 115F in the stowed configuration similar to FIG. 20A. Here, three laparoscopic arms 115R are stowed beneath a first side of the table top 160 (e.g., the head of table top 160) and two robotic arms, including one laparoscopic arm 115R and one endoscopic arm 115F are stowed beneath the opposite side of the table top 160 (e.g., the foot of the table top 160). As seen in FIG. 21A, deployment system 125 can allow stowage of the robotic arms 115R, 115F with little to no footprint beyond the perimeter of the table top 160 when viewed from above.

FIG. 21B is a top view of deployment system 125 with laparoscopic arms 115R deployed in a 2+2 configuration, and endoscopic arm 115F positioned at the head of table top 160. Endoscopic arm 115F can remain stowed for a purely laparoscopic procedure, such as a Roux-en-Y Gastric Bypass (RYGB), or can be deployed at the head of the table top 160 for an upper GI combined endoscopic and laparoscopic procedure.

FIG. 21C is a top view of deployment system 125 with laparoscopic arms 115R deployed in a 3+1 configuration, and endoscopic arm 115F positioned at the foot of table top 160. Endoscopic arm 115F can remain stowed for a purely laparoscopic procedure, such as a Low Anterior Resection (LAR), or can be deployed at the foot of the table top 160 for a lower GI combined endoscopic and laparoscopic procedure. As seen in FIGS. 21B-21C, the arm carriage 101 that carries endoscopic arm can traverse the arm support 163 to the head or the foot of the table, allowing a single endoscopic arm 115F to access the head or foot of the surgical table and patient as appropriate for a given procedure. Similarly, one or more of the arm carriages 101 carrying a laparoscopic arm 115R can traverse the arm support 163 to either lateral side of the table, allowing the laparoscopic arms 115R to be readily placed in 2+2 and 3+1 configurations as appropriate for a given procedure.

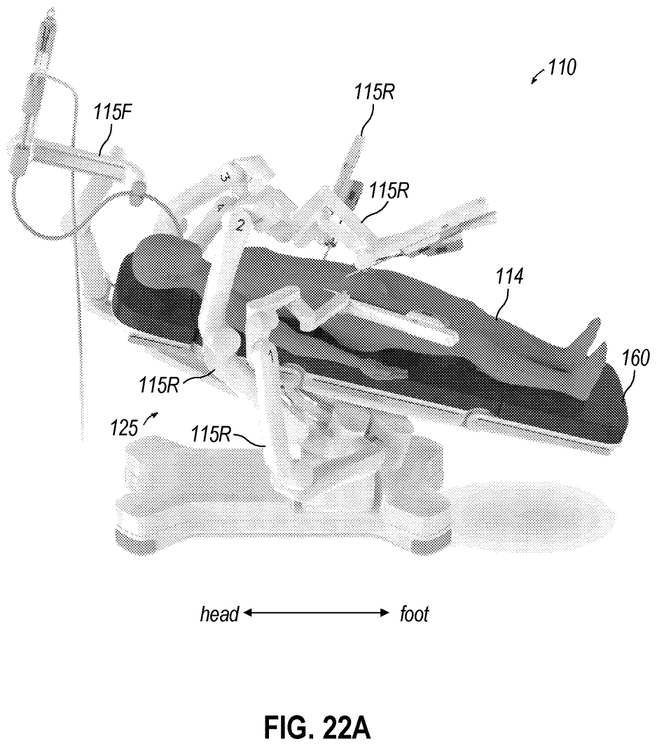

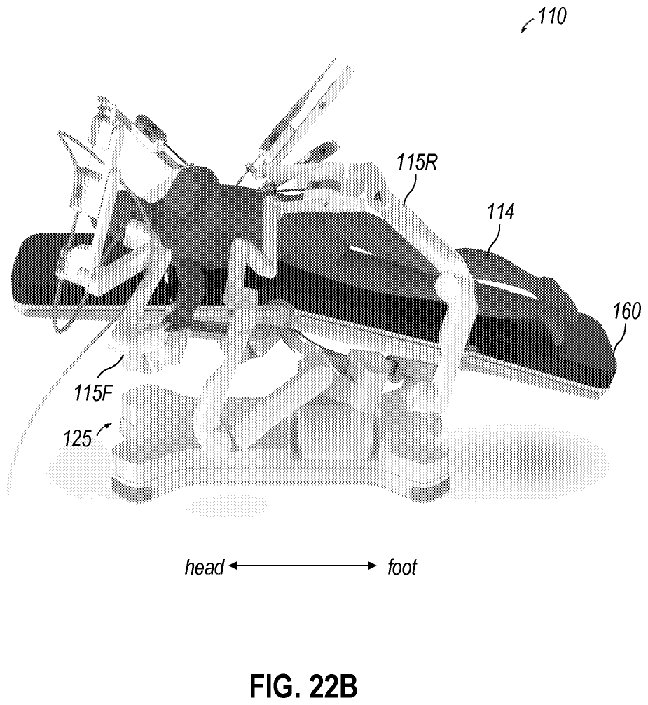

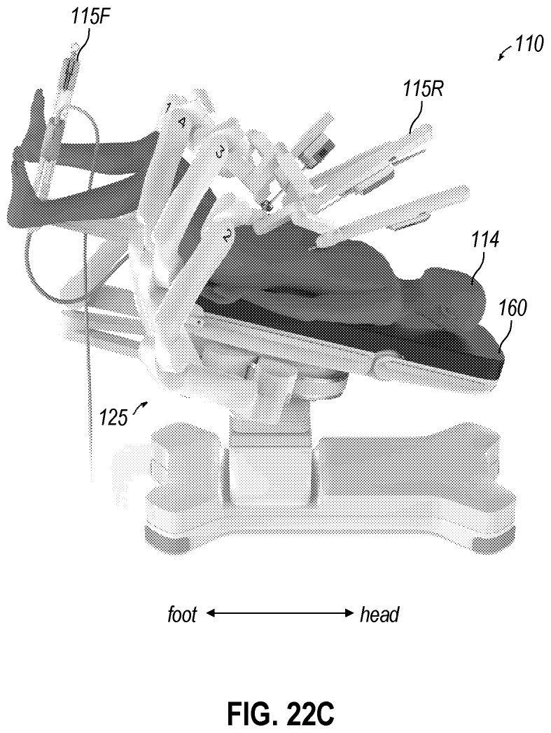

FIGS. 22A-22C depict perspective views of deployment system 125 in use performing procedures on patient 114 supported on table top 160.

FIG. 22A depicts deployment system 125 in use for an esophageal procedure. Endoscopic arm 115F is positioned at the head of the table to access a mouth of the patient with flexible instrument(s), and laparoscopic arms 115R positioned in a 2+2 configuration to access the abdomen of the patient 114.

FIG. 22B depicts deployment system 125 in use for a thoracic procedure. Endoscopic arm 115F is positioned to access the head of patient 114 from a lateral side of the table, and laparoscopic arms or rigid instrument arms 115R are positioned in a 2+2 configuration to access the chest of the patient 114.

FIG. 22C depicts deployment system 125 in use for a perineal procedure. Endoscopic arm 115F is positioned at the foot of the table to access the perineum of patient 114, and laparoscopic arms 115R are positioned in a 3 +1 configuration to access the abdomen of the patient 114. FIG. 22C also depicts the surgical table with a footboard of table top 160 removed to facilitate access to the perineum by endoscopic arm 115F.

Various principles of this technology are described with reference to laparoscopic procedures, where surgical instruments are introduced to a patient's abdomen through laparoscopic incisions, and endoluminal procedures, where surgical instruments are introduced through natural orifices. In some variations, a configuration of a surgical system and/or a method of use can involve various types of procedures, anatomical locations, and/or anatomical openings for introducing instruments into a body. Various aspects of the subject matter described herein may be applied to, for instance, purely laparoscopic procedures, purely endoluminal procedures, purely thoracic procedures, open procedures, procedures involving only flexible instruments, procedures involving only rigid instruments, and/or procedures involving any combination of two or more of these approaches.

Various principles of this technology are described with reference to rigid and flexible instrumentation, where, for instance, rigid instruments are introduced through incisions or laparoscopic ports, and flexible instruments are introduced through natural orifices or endoluminal access points. In various configurations, any one or more of these instruments may be flexible or rigid. For instance, in some variations, one or more rigid instruments or rigid scopes may be introduced through a natural orifice. Additionally or alternatively, in some variations, more flexible instruments or flexible scopes may be introduced through an incision or a laparoscopic port.

Various examples disclosed herein describe usage of a surgical system to perform a procedure on a patient, wherein instruments are inserted into a body of the patient. In various configurations, the system may be used, for instance, in educational or lab settings, where a body portion of a model, cadaver, animal, or inanimate object is placed upon the headrest. Such methods may be useful for surgeon training, product testing, development applications, or the like. Accordingly, it will be understood that methods described herein are not limited to medical procedures performed on a human body but can be implemented on bodies or objects that are not part of a live patient or human.

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for illustrative purposes. It is contemplated that the various teachings herein may be arranged and applied in numerous other ways. It is also contemplated that some variations may omit certain features referred to in the examples below. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example 1: A robotic system comprising: an arm support comprising a track; a cable path extending alongside the track; an arm carriage movably mounted to the track such that the arm carriage moves along the track; a robotic arm mounted to the arm carriage such that the robotic arm moves with the arm carriage along the track; a spool rotatably mounted to the arm carriage such that the spool rotates relative to the arm carriage as the arm carriage moves along the track; an electrical cable having a first portion attached to the spool and a second portion attached to the cable path such that the electrical cable winds onto the spool from the cable path or pays out from the spool onto the cable path as the arm carriage moves along the track; and a slip ring mounted to the arm carriage, the slip ring electrically and rotatably coupling the electrical cable to the robotic arm.

Example 2: The robotic system of Example 1, further comprising: a second arm carriage movably mounted to the track such that the second arm carriage moves along the track; a second robotic arm mounted to the second arm carriage such that the second robotic arm moves with the second arm carriage along the track; a second spool rotatably mounted to the second arm carriage such that the second spool rotates relative to the second arm carriage as the second arm carriage moves along the track; and a second electrical cable having a first portion attached to the second spool and a second portion attached to the cable path such that the second electrical cable pays out from the second spool onto the cable path and onto the electrical cable as the arm carriage moves along the track.

Example 3: The robotic system of Example 1 or Example 2, the arm support being ring-shaped such that the track follows a circular path.

Example 4: The robotic system of any one of Examples 1-3, further comprising: a ring gear fixedly coupled to the arm support alongside the track; and a carriage actuator mounted to the arm carriage and operatively coupled to the ring gear via a pinion gear, the carriage actuator operable to drive movement of the arm carriage along the track via actuation of the pinion gear.

Example 5: The robotic system of any one of Examples 1-4, further comprising: an encoder mounted to the arm carriage and operable to detect a position of the arm carriage along the arm support.

Example 6: The robotic system of any one of Examples 1-5, the encoder comprising: a coarse encoder operable to detect a current gear tooth among a plurality of gear teeth extending alongside the track; and a fine encoder operable to detect a position relative to the current gear tooth.

Example 7: The robotic system of any one of Examples 1-6, further comprising: a brake mounted to the arm carriage and operable to inhibit movement of the arm carriage along the arm support, the brake comprising: a movable component movable against an engagement surface to inhibit the movement of the arm carriage along the arm support, the engagement surface extending alongside the track; a brake spring operably coupled to the movable component and biased to urge the movable component against the engagement surface; and a brake actuator operably coupled to the movable component and operable to disengage the brake.

Example 8: The robotic system of any one of Examples 1-7, the movable component comprising a wedge.

Example 9: The robotic system of any one of Examples 1-8, the movable component comprising a set of interleaved plates interleaved with a complementary set of interleaved plates of the engagement surface.

Example 10: The robotic system of any one of Examples 1-9, the robotic arm being an endoscopic arm, the robotic system further comprising: a plurality of arm carriages supporting a plurality of laparoscopic arms, the arm carriage and the plurality of arm carriages movable to a plurality of configurations along the arm support, the plurality of configurations including: a first configuration, where the endoscopic arm is positioned at a foot of a surgical table and at least one of the laparoscopic arms is positioned at a lateral side of the surgical table; and a second configuration, where the endoscopic arm is positioned at a head of the surgical table opposite the foot of the surgical table and at least one of the laparoscopic arms is positioned at a lateral side of the surgical table.

Example 11: A robotic system comprising: a support; a first carriage mounted to the support and configured to move along the support; a first cable coupled to the first carriage and configured to pay out onto a cable path as the first carriage moves along the support; a second carriage mounted to the support and configured to move along the support; and a second cable coupled to the second carriage and configured to pay out onto the first cable as the second carriage moves along the support.

Example 12: The robotic system of Example 11, further comprising: a third carriage mounted to the support and configured to move along the support; and a third cable coupled to the third carriage and configured to pay out onto the second cable as the second carriage moves along the support.

Example 13: The robotic system of Example 11 or Example 12, the first carriage comprising a first spool and a first slip ring, the first cable being wrapped around the first spool and electrically coupled to the first slip ring, and the second carriage comprising a second spool and a second slip ring, the second cable being wrapped around the second spool and electrically coupled to the second slip ring.

Example 14: The robotic system of any one of Examples 11-13, the first carriage comprising a first spring operatively coupled to the first spool and configured to maintain tension on the first cable, the second carriage comprising a second spring operatively coupled to the second spool and configured to maintain tension on the second cable.

Example 15: The robotic system of any one of Examples 11-14, the cable path extending around a circumference of a cylinder.

Example 16: The robotic system of any one of Examples 11-15, the first carriage being configured to support a first robotic arm, and the second carriage being configured to support a second robotic arm.

Example 17: A robotic system comprising: a surgical table configured to support a patient; an arm support coupled to the surgical table; a plurality of robotic arms coupled to the arm support, the plurality of robotic arms including an endoscopic arm and a plurality of laparoscopic arms movable along the arm support to a plurality of configurations, the plurality of configurations including: a first configuration, where the endoscopic arm is positioned at a foot of the surgical table and at least one of the laparoscopic arms is positioned at a lateral side of the surgical table; and a second configuration, where the endoscopic arm is positioned at a head of the surgical table opposite the foot of the surgical table and at least one of the laparoscopic arms is positioned at a lateral side of the surgical table.

Example 18: The robotic system of Example 17, the first configuration having two of the laparoscopic arms positioned at a first lateral side of the surgical table and two of the laparoscopic arms positioned at a second lateral side of the surgical table opposite the first lateral side.

Example 19: The robotic system of Example 17 or Example 18, the second configuration having three of the laparoscopic arms positioned at a first lateral side of the surgical table and one of the laparoscopic arms positioned at a second lateral side of the surgical table opposite the first lateral side.

Example 20: The robotic system of any one of Examples 17-19, the arm support encircling a column of the surgical table, the robotic system further comprising: a plurality of arm carriages movably coupled to the arm support, the plurality of arm carriages supporting the plurality of robotic arms, the plurality of arm carriages being configured to independently traverse the arm support to place the plurality of robotic arms in the plurality of configurations.

Example 21: A robotic system comprising: an arm support comprising a track; a ring gear extending alongside the track; an arm carriage movably mounted to the track; a robotic arm mounted to the arm carriage; and a carriage actuator mounted to the arm carriage and operably coupled to the ring gear via a pinion gear, the carriage actuator operable to drive movement of the arm carriage along the track via actuation of the pinion gear.

Example 22: A robotic system comprising: an arm support comprising a track; an engagement surface extending alongside the track; an arm carriage movably mounted to the track; a robotic arm mounted to the arm carriage; and a brake mounted to the arm carriage, the brake comprising: a movable component; a brake spring operatively coupled to the movable component, the brake spring biased to urge the movable component against the engagement surface to thereby inhibit movement of the arm carriage relative to the arm support; and a brake actuator operatively coupled to the movable component, the brake actuator operable to urge the movable component away from the engagement surface to thereby disengage the brake.

Use of “or” is intended in the inclusive rather than exclusive sense, unless explicitly stated otherwise or the context clearly dictates otherwise. Thus, for example, reference to “A” or “B” can encompass “A” only, “B” only, or both “A” and “B.” As another example, reference to “A, B, or C” can encompass “A” only, “B” only, “C” only, or any combination of two or more of “A” or “B” or “C.” Accordingly, the term “or” should be generally understood as equivalent to “and/or” unless stated otherwise or the context clearly dictates to the contrary.

It should be appreciated that any specific order of steps shown or described herein is illustrative in nature and should not be construed as required unless explicitly stated or the context clearly dictates otherwise. Thus, for example, with respect to any processes or methods herein, any two or more steps or stages in a method or process may performed serially or in parallel, in any combination, and may be performed in any order, unless explicitly stated or the context clearly dictates otherwise.

In some instances, relative positions or orientations are used, such as top, bottom, upper, lower, forward, backward, front, rear, left, right, up down, horizontal, vertical, longitudinal, lateral, or the like. These terms may be used to refer to an arbitrary frame of reference or a frame of reference shown in the drawings, for purposes of explanation or to demonstrate the relative spatial configurations associated with various elements. These terms should not be understood to require any particular gravitational or other frame of reference unless explicitly stated or the context clearly dictates otherwise.

To the extent any headings are used through this description, these headings are used for convenience only and should not be construed as limit the scope of disclosure or the description under a heading to only the topic associated with the heading in anyway.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various examples, configurations, or embodiments of the present technology, further adaptations of the systems or methods described herein may be accomplished by appropriate modifications by one of ordinary skill in the art without departing from the scope of the technology described herein. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the claimed subject matter should be considered in terms of the following claims and is understood not to be limited to the details of structure and operation shown and described in the specification and drawings.

1. A robotic system comprising:
an arm support comprising a track;
a cable path extending alongside the track;
an arm carriage movably mounted to the track such that the arm carriage moves along the track;
a robotic arm mounted to the arm carriage such that the robotic arm moves with the arm carriage along the track;
a spool rotatably mounted to the arm carriage such that the spool rotates relative to the arm carriage as the arm carriage moves along the track;
an electrical cable having a first portion attached to the spool and a second portion attached to the cable path such that the electrical cable winds onto the spool from the cable path or pays out from the spool onto the cable path as the arm carriage moves along the track; and
a slip ring mounted to the arm carriage, the slip ring electrically and rotatably coupling the electrical cable to the robotic arm.

2. The robotic system of claim 1, further comprising:
a second arm carriage movably mounted to the track such that the second arm carriage moves along the track;
a second robotic arm mounted to the second arm carriage such that the second robotic arm moves with the second arm carriage along the track;
a second spool rotatably mounted to the second arm carriage such that the second spool rotates relative to the second arm carriage as the second arm carriage moves along the track; and
a second electrical cable having a first portion attached to the second spool and a second portion attached to the cable path such that the second electrical cable pays out from the second spool onto the cable path and onto the electrical cable as the arm carriage moves along the track.

3. The robotic system of claim 1, the arm support being ring-shaped such that the track follows a circular path.

4. The robotic system of claim 1, further comprising:
a ring gear fixedly coupled to the arm support alongside the track; and
a carriage actuator mounted to the arm carriage and operatively coupled to the ring gear via a pinion gear, the carriage actuator operable to drive movement of the arm carriage along the track via actuation of the pinion gear.

5. The robotic system of claim 1, further comprising:
an encoder mounted to the arm carriage and operable to detect a position of the arm carriage along the arm support.

6. The robotic system of claim 5, the encoder comprising:
a coarse encoder operable to detect a current gear tooth among a plurality of gear teeth extending alongside the track; and
a fine encoder operable to detect a position relative to the current gear tooth.

7. The robotic system of claim 1, further comprising:
a brake mounted to the arm carriage and operable to inhibit movement of the arm carriage along the arm support, the brake comprising:
a movable component movable against an engagement surface to inhibit the movement of the arm carriage along the arm support, the engagement surface extending alongside the track;
a brake spring operably coupled to the movable component and biased to urge the movable component against the engagement surface; and
a brake actuator operably coupled to the movable component and operable to disengage the brake.

8. The robotic system of claim 7, the movable component comprising a wedge.

9. The robotic system of claim 7, the movable component comprising a set of interleaved plates interleaved with a complementary set of interleaved plates of the engagement surface.

10. The robotic system of claim 1, the robotic arm being an endoscopic arm, the robotic system further comprising:
a plurality of arm carriages supporting a plurality of laparoscopic arms, the arm carriage and the plurality of arm carriages movable to a plurality of configurations along the arm support, the plurality of configurations including:
a first configuration, where the endoscopic arm is positioned at a foot of a surgical table and at least one of the laparoscopic arms is positioned at a lateral side of the surgical table; and
a second configuration, where the endoscopic arm is positioned at a head of the surgical table opposite the foot of the surgical table and at least one of the laparoscopic arms is positioned at a lateral side of the surgical table.

11. A robotic system comprising:
a support;
a first carriage mounted to the support and configured to move along the support;
a first cable coupled to the first carriage and configured to pay out onto a cable path as the first carriage moves along the support;
a second carriage mounted to the support and configured to move along the support; and
a second cable coupled to the second carriage and configured to pay out onto the first cable as the second carriage moves along the support.

12. The robotic system of claim 11, further comprising:
a third carriage mounted to the support and configured to move along the support; and
a third cable coupled to the third carriage and configured to pay out onto the second cable as the second carriage moves along the support.

13. The robotic system of claim 11,
the first carriage comprising a first spool and a first slip ring, the first cable being wrapped around the first spool and electrically coupled to the first slip ring, and
the second carriage comprising a second spool and a second slip ring, the second cable being wrapped around the second spool and electrically coupled to the second slip ring.

14. The robotic system of claim 13,
the first carriage comprising a first spring operatively coupled to the first spool and configured to maintain tension on the first cable,
the second carriage comprising a second spring operatively coupled to the second spool and configured to maintain tension on the second cable.

15. The robotic system of claim 11, the cable path extending around a circumference of a cylinder.

16. The robotic system of claim 11, the first carriage being configured to support a first robotic arm, and the second carriage being configured to support a second robotic arm.

17-22. (canceled)
